(19) United States
(12) Patent Application Publication
(10) Pub. No.: US 2006/0128653 A1
(43) Pub. Date: Jun. 15, 2006

(54) Pharmaceutical formulation of decitabine

(76) Inventors: Chunlin Tang, Walnut Creek, CA (US); Rajashree Joshi-Hangal, Pleasanton, CA (US)

Correspondence Address: WILSON SONSINI GOODRICH & ROSATI, 650 PAGE MILL ROAD, PALO ALTO, CA, 94304-1050, US

(21) Appl. No.: 11/009,540

(22) Filed: Dec. 10, 2004

(51) Int. Cl. A61K 31/7072; A61K 31/724

(52) U.S. Cl. 514/49; 514/58

(57) ABSTRACT

The present invention provides pharmaceutical formulations of decitabine or 5-aza-2′-deoxycytidine as well as methods of manufacturing the formulations. In particular, decitabine is formulated with a cyclodextrin compound to stabilize and/or enhance solubility of the drug. Kits and methods for using the pharmaceutical formulations are also provided, including methods of administering decitabine to treat conditions or diseases, such as cancer and hematological disorders.

### FIELD OF THE INVENTION

This invention relates generally to pharmaceutical formulations of cytidine analogs or derivatives, and more particularly relates to aqueous formulations of cytidine analogs and derivatives such as decitabine containing a cyclodextrin compound, and methods of preparing and using the pharmaceutical formulations for treating various diseases and conditions, such as cancer and hematological disorders.

### BACKGROUND OF THE INVENTION

A few azacytosine nucleosides, such as 5-aza-2′-deoxycytidine (also called decitabine) and 5-azacytidine (also called azacitidine), have been developed as antagonist of its related natural nucleoside, 2′-deoxycytidine and cytidine, respectively. The only structural difference between azacytosine and cytosine is the presence of a nitrogen at position 5 of the cytosine ring in azacytosine as compared to a carbon at this position for cytosine.

Two isomeric forms of decitabine can be distinguished. The β-anomer is the active form. The modes of decomposition of decitabine in aqueous solution are (a) conversion of the active β-anomer to the inactive α-anomer (Pompon et al. (1987) J. Chromat. 388:113-122); (b) ring cleavage of the aza-pyrimidine ring to form N-(formylamidino)-N′-β-D-2′-deoxy-(ribofuranosy)-urea (Mojaverian and Repta (1984) J. Pharm. Pharmacol. 36:728-733); and (c) subsequent formation of guanidine compounds (Kissinger and Stemm (1986) J. Chromat. 353:309-318).

Decitabine possesses multiple pharmacological characteristics. At a molecular level, it is S-phase dependent for incorporation into DNA. At a cellular level, decitabine can induce cell differentiation and exert hematological toxicity. Despite having a short half-life in vivo, decitabine has an excellent tissue distribution (Van Groeningen C J, et al. (1986) Cancer Res. 46:4831-4836; and Chabot G G and Momparler R L (1990) Proceedings of the Workshop on 5-aza-2′-deoxycytidine (Momparler R L and De Vos D. eds), PCH Publication, pp. 105-115).

One of the functions of decitabine is its ability to specifically and potently inhibit DNA methylation. Methylation of cytosine to 5-methylcytosine occurs at the level of DNA. Inside the cell, decitabine is first converted into its active form, the phosphorylated 5-aza-deoxycytidine, by deoxycytidine kinase which is primarily synthesized during the S phase of the cell cycle. The affinity of decitabine for the catalytical site of deoxycytidine kinase is similar to the natural substrate, deoxycytidine. Momparler et al. (1985) 30:287-299. After conversion to its triphosphate form by deoxycytidine kinase, decitabine is incorporated into replicating DNA at a rate similar to that of the natural substrate, dCTP. Bouchard and Momparler (1983) Mol. Pharmacol. 24:109-114.

Incorporation of decitabine into the DNA strand has a hypomethylation effect by reversal of aberrant hypermethylation characteristic of cancer as a disease. Each class of differentiated cells has its own distinct methylation pattern. After chromosomal duplication, in order to conserve this pattern of methylation, the 5-methylcytosine on the parental strand serves to direct methylation on the complementary daughter DNA strand. Substituting the carbon at the 5 position of the cytosine for a nitrogen interferes with this normal process of DNA methylation. The replacement of 5-methylcytosine with decitabine at a specific site of methylation produces an irreversible inactivation of DNA methyltransferase, presumably due to formation of a covalent bond between the enzyme and decitabine. Juttermann et al. (1994) Proc. Natl. Acad. Sci. USA 91:11797-11801. By specifically inhibiting DNA methyltransferase, the enzyme required for methylation, the aberrant methylation of the tumor suppressor genes could be prevented.

Decitabine is commonly supplied as a sterile lyophilized powder for injection, together with buffering salt, such as potassium dihydrogen phosphate, and pH modifier, such as sodium hydroxide. For example, decitabine is supplied by SuperGen, Inc., as lyophilized powder packed in 20 mL glass vials, containing 50 mg of decitabine, monobasic potassium dihydrogen phosphate, and sodium hydroxide. When reconstituted with 10 mL of sterile water for injection, each mL contain 5 mg of decitabine, 6.8 mg of KH₂PO₄, and approximately 1.1 mg NaOH. The pH of the resulting solution is 6.5-7.5. The reconstituted solution can be further diluted to a concentration of 1.0 or 0.1 mg/mL in cold infusion fluids, i.e., 0.9% Sodium Chloride; or 5% Dextrose; or 5% Glucose; or Lactated Ringer's. The unopened vials are typically stored at room temperature (2-25° C.; 32-77° F.), in the original package.

Decitabine is most typically administrated to patients by injection, such as by a bolus I.V. injection, continuous I.V. infusion, or I.V. infusion. Similar to decitabine, azacitidine is also formulated as aqueous solution and delivered to patients intravenously. According to clinical studies of azacitidine, longer or continuous infusions were more effective than shorter ones. Santini et al. (2001) Ann. Int. Med. 134:573-588. However, the length of I.V. infusion is limited by the decomposition/instability of decitabine or azacitidine and low solubility of the drugs in aqueous solutions. The present invention provides innovative solutions to such problems.

### SUMMARY OF THE INVENTION

The present invention provides formulations of cytidine analogs or derivatives (e.g., decitabine and 5-azacytidine), kits and methods of using the cytidine analogs or derivatives for preventing or treating various diseases or conditions. According to the invention, the cytidine analog/derivative is formulated with a cyclodextrin compound, preferably solvated in aqueous solvent. The innovative approach circumvents problems associated with the current clinical aqueous formulations of decitabine or 5-azacytidine in phosphate buffer, such as chemical instability, inconvenient storage and transportation, and discomfort of patients due to cold infusions.

In one aspect of the invention, a pharmaceutical composition is provided, comprising: a cytidine analog or derivative and a cyclodextrin compound, preferably in solid form. The composition may further comprise buffer salt, acid, alkaline, and/or excipient. Upon dissolving in aqueous solution, the cytidine analog/derivative forms complex with the cyclodextrin compound. The pharmaceutical composition may be in various dosage forms suitable for delivering into an animal subject orally, parenterally, topically, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, or intrathecally. Optionally, the pharmaceutical composition may be solvated with aqueous solvent and then administered to the subject intravenously, intramuscularly, or subcutaneously.

In another aspect of the invention, an aqueous pharmaceutical formulation is provided for administering a cytidine analog/derivative to a patient. In one embodiment, the formulation comprises decitabine solvated in a solvent that comprises a cyclodextrin compound.

The cytidine analog/derivative is preferably decitabine, 5-azacytidine, 5-aza-2′-deoxy-2′,2′-difluorocytidine, 5-aza-2′-deoxy-2′-fluorocytidine, 2′-deoxy-2′,2′-difluorocytidine (also called gemcitabine), or cytosine 1-β-D-arabinofuranoside (also called ara-C).

The cytidine analog/derivative may be in the form of salt, preferably pharmaceutically-acceptable salt. Examples of the salt of cytidine analog include, but are not limited to pharmaceutically-acceptable salts prepared from an inorganic acid or an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, arylaliphatic, heterocyclic, carboxylic and sulfonic classes of organic acids, examples of which are formic, acetic, propionic, succinic, glycolic, gluconic, maleic, embonic (pamoic), methanesulfonic, ethanesulfonic, 2-hydroxyethanesulfonic, pantothenic, benzenesulfonic, toluenesulfonic, sulfanilic, mesylic, cyclohexylaminosulfonic, stearic, algenic, β-hydroxybutyric, malonic, galactic, and galacturonic acid. Preferably, the acid is selected from the group consisting of hydrochloric, L-lactic, acetic, phosphoric, (+)-L-tartaric, citric, propionic, butyric, hexanoic, L-aspartic, L-glutamic, succinic, EDTA, maleic, methanesulfonic acid, HBr, HF, HI, nitric, nitrous, sulfuric, sulfurous, phosphorous, perchloric, chloric, chlorous acid, carboxylic acid, sulfonic acid, ascorbic, carbonic, and fumaric acid. In particular, the sulfonic acid is selected from the group consisting of ethanesulfonic, 2-hydroxyethanesulfonic, and toluenesulfonic acid.

The cyclodextrin compound may be crystalline and amorphous. Preferably the cyclodextrin compound is amorphous α-, β- or γ-cyclodextrin, more preferably alkylated or hydroxyalkyl cyclodextrin selected from the group consisting of hydroxypropyl, hydroxyethyl, glucosyl, maltosyl and maltotriosyl derivatives of β-cyclodextrin, carboxyamidomethyl-β-cyclodextrin, carboxymethyl-β-cyclodextrin, hydroxypropyl-β-cyclodextrin and diethylamino-β-cyclodextrin.

Optionally, the cyclodextrin compound is a sulfoalkylether cyclodextrin derivative, preferably mono-, tetra or hepta-substituted β-cyclodextrin sulfobutyl ether sodium salt, and more preferably β-cyclodextrin sulfobutyl ether, 7 sodium salt (CAPTISOL®).

According to the embodiment, the amount of the cytidine analog/derivative in the pharmaceutical formulation is between 0.1 and 200 mg per ml of solvent, optionally between 1 and 100, between 2 mg and 50 mg, 5 mg and 30 mg, between 10 mg and 25 mg per ml of the solvent.

The ratio of the weight of the cytidine analog/derivative to the weight of cyclodextrin compound maybe in a range between 1:1 and 1:5000, optionally between 1:10 and 1:300, between 1:1 and 1:200, or between 1:5 and 1:50.

Optionally, the pharmaceutical formulation may comprise at least 60%, 70%, 80%, 90%, 95% or 99% v/v water. The concentration of the cyclodextrin compound in the aqueous pharmaceutical formulation is between 0.1% and 80% w/w, optionally between 1% and 60% w/w, optionally between 5% and 50% w/w, optionally between 10% and 40% w/w, or optionally between 20% and 40% w/w.

Also optionally, the pharmaceutical formulation may further comprise propylene glycol, glycerin, and/or polyethylene glycol (PEG) such as PEG300, PEG400 and PEG1000.

Also according to the embodiment, the pharmaceutical composition may further comprise an acidifying agent added to the formulation in a proportion such that the formulation has a resulting pH between about 4 and 8. Adding an acidifying agent to control the pH of the formulation is believed to facilitate ready dissolution of the cytidine analog/derivative in the solvent and enhance long-term stability of the formulation.

The acidifying agent may be an organic acid. Examples of organic acid include, but are not limited to, ascorbic acid, citric acid, tartaric acid, lactic acid, oxalic acid, formic acid, benzene sulphonic acid, benzoic acid, maleic acid, glutamic acid, succinic acid, aspartic acid, diatrizoic acid, and acetic acid. The acidifying agent may also be an inorganic acid, such as hydrochloric acid, sulphuric acid, phosphoric acid, and nitric acid.

In a variation, the acidifying agent is ascorbic acid at a concentration of 0.01-0.2 mg/ml of the solvent, optionally 0.04-0.1 mg/ml or 0.03-0.07 mg/ml of the solvent.

The pH of the pharmaceutical formulation may be adjusted to be between pH 4 and pH 8, preferably between pH 5 and pH 7, and more preferably between pH 5.5 and pH 6.8.

The pharmaceutical formulation may optionally further include an excipient added in an amount sufficient to enhance the stability of the composition, maintain the product in solution, or prevent side effects (e.g., potential ulceration, vascular irritation or extravasation) associated with the administration of the inventive formulation. Examples of excipients include, but are not limited to, mannitol, sorbitol, lactose, and dextrose.

The pharmaceutical formulation is preferably at least 80%, 90%, 95% or more stable upon storage at 2-8° C. for 5, 10, 15, 24 hours, or 2, 4, 7, 14, 21, 28 or more days. The pharmaceutical formulation is also preferably at least 80%, 90%, 95% or more stable upon storage at 20-25° C. for 5, 10, 15, 24 hours, or 2, 5, 7, 14, 21, 28 or more days.

In another embodiment, the pharmaceutical formulation is prepared by the act comprising: dissolving a cytidine analog/derivative and a cyclodextrin compound in an aqueous solvent that comprises at least 60% v/v water, optionally at least 70%, 80%, 90%, 95% or 99% v/v water. The aqueous solvent may further comprise buffer salt such as potassium phosphate dissolved therein.

According to the embodiment, the act may further comprise: adding an acidifying agent to the solution containing the cytidine analog/derivative and the solvent such that pH of the resulting solution is between pH 4 and pH 8, preferably between pH 5 and pH 7, and more preferably between pH 5.5 and pH 6.8.

The pharmaceutical formulation may also optionally comprise one or more therapeutic agents other than the cytidine analog/derivative. For example, the pharmaceutical formulation may optionally further comprise a therapeutic agent selected from the group consisting of anti-neoplastic agents, alkylating agents, agents that are members of the retinoids superfamily, hormonal agents, plant-derived agents, biologic agents, interleukins, interferons, cytokines, immuno-modulating agents, and monoclonal antibodies.

In another aspect of the invention, a sterilized vessel is provided for administering a cytidine analog/derivative to a host in need thereof. In one embodiment, the sterilized vessel comprises a pharmaceutical formulation according to the present invention. The vessel, for example, may be a vial, syringe, or ampoule. The vessel may come in different sizes. For example, the vessel may comprise between 1 and 50, 1 and 25, 1 and 20 or 1 and 10 ml of the pharmaceutical formulation.

In yet another aspect of the invention, a kit is provided for administering a cytidine analog/derivative to a host in need thereof. In one embodiment, the kit comprises a cytidine analog/derivative in a solid, preferably powder, or more preferably lyophilized powder form, and an aqueous diluent that comprises a cyclodextrin compound. Mixing of the solid analog/derivative and the diluent results in the formation of a pharmaceutical formulation according to the present invention.

For example, the kit may comprise a first container containing a cytidine analog/derivative and a cyclodextrin compound in a solid, preferably powder, or more preferably lyophilized powder form; and a second container containing an aqueous diluent, wherein adding the diluent to the solid compounds results in the formation of a pharmaceutical formulation for administering the cytidine analog/derivative.

Optionally, the kit may comprise a first container containing a cytidine analog/derivative in a solid form; and a second container containing a cyclodextrin compound dissolved in an aqueous diluent, wherein adding the diluent to the solid compound results in the formation of a pharmaceutical formulation for administering the cytidine analog/derivative.

Also optionally, the kit may comprise a first container containing a cytidine analog/derivative in a solid form; a second container containing a cyclodextrin compound in a solid form; and a third container containing an aqueous diluent, wherein adding the diluent to the solid cyclodextrin compound results in the formation of an aqueous solution which is then added to the solid cytidine analog/derivative to produce a pharmaceutical formulation for administering the cytidine analog/derivative.

Mixing the solid cytidine analog/derivative and diluent may optionally form a pharmaceutical formulation that comprises between 0.1 and 200 mg cytidine analog/derivative per ml of the diluent, optionally between 1 and 100, between 2 mg and 50 mg, 5 mg and 30 mg, between 10 mg and 25 mg per ml of the solvent.

The kit may optionally further include instructions. The instructions may describe how the solid compound(s) and the diluent should be mixed to form a pharmaceutical formulation. The instructions may also describe how to administer the resulting pharmaceutical formulation to a patient. It is noted that the instructions may optionally describe the administration methods according to the present invention.

The diluent and cytidine analog/derivative may be contained in separate vessels. The vessels may come in different sizes. For example, the vessel may comprise between 1 and 50, 1 and 25, 1 and 20, or 1 and 10 ml of the diluent.

In yet another aspect of the invention, a method is provided for administering a cytidine analog/derivative to a host in need of, such as a patient suffering from a disease that is sensitive to the treatment with the cytidine analog/derivative. The pharmaceutical formulation of the present invention may be administered orally, parenterally, topically, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, or intrathecally. Preferably, the pharmaceutical formulation is administered intravenously, intramuscularly, or subcutaneously.

In one embodiment, the method comprises: administering to the patient a therapeutically effective amount of a cytidine analog/derivative in a pharmaceutical formulation according to the present invention. Optionally, the pharmaceutical formulation comprises at least 60%, 70%, 80%, 90%, 95% or 99% v/v water prior to administration to the patient.

The method may further comprise administering a therapeutic agent other than the cytidine analog/derivative in combination with the pharmaceutical formulation. The therapeutic agent may be selected from the group consisting of anti-neoplastic agents, alkylating agents, agents that are members of the retinoids superfamily, hormonal agents, plant-derived agents, biologic agents, interleukins, interferons, cytokines, immuno-modulating agents, and monoclonal antibodies.

In another embodiment, the method comprises: taking a pharmaceutical formulation comprising a cyclodextrin and between 0.1 and 200 mg/mL cytidine analog/derivative solvated in an aqueous solvent; diluting the pharmaceutical formulation with an aqueous solution; and administering the resulting diluted pharmaceutical formulation; wherein the dilution is performed 10 hr, 2 hr, 1 hr, 30 min, 10 min, 5 min or less before administration.

In yet another embodiment, the method comprises: taking a pharmaceutical formulation comprising a cyclodextrin and between 0.1 and 200 mg cytidine analog/derivative solvated in an aqueous solvent; admixing aliquots of the pharmaceutical formulation with an aqueous solution at ambient temperature; and infusing the resulting solution into the patient's body. A Y connector is optionally used to admix the aliquots of the pharmaceutical formulation with the aqueous solution at ambient temperature. This allows the infusion to be optionally performed over a period of 3, 4, 5 or more hours. Such a mode of administration is believed to cause less discomfort in the patient and allow slower and longer infusion time than that needed for administering decitabine (or 5-azacytidine) formulated in the conventional ways which require decitabine be reconstituted in WFI and further diluted with cold infusion fluid.

Related to the kit, a method is also provided that comprises mixing a cytidine analog/derivative and a cyclodextrin compound that are in a solid, preferably powder form with a diluent to form a pharmaceutical formulation, and administering the pharmaceutical formulation to a patient.

In another embodiment, the method comprises mixing a cytidine analog/derivative that is in a solid, preferably powder form with a diluent comprising a cyclodextrin compound to form a pharmaceutical formulation, and administering the pharmaceutical formulation to a patient.

Advantageously, the pharmaceutical formulation may be formed by mixing the cytidine analog/derivative with the diluent shortly prior to administration to a patient (e.g., within one day, or even 6, 5, 4, 3, 2 or 1 hours or less before administration). This reduces decomposition of the cytidine analog/derivative. Optionally, as described herein, the pharmaceutical formulation may be administered by admixing aliquots of the pharmaceutical formulation with an aqueous solution (e.g., infusion fluid); and infusing the resulting solution into the patient's body, optionally with a Y connector as also described herein.

Also according to the present invention, a method is provided for treating a disease associated with undesirable cell proliferation in a subject. The method comprises administering to the subject in need thereof a cytidine analog/derivative formulated with a cyclodextrin compound. The disease may be benign tumors, cancer, hematological disorders, atherosclerosis, insults to body tissue due to surgery, abnormal wound healing, abnormal angiogenesis, diseases that produce fibrosis of tissue, repetitive motion disorders, disorders of tissues that are not highly vascularized, or proliferative responses associated with organ transplants. In particular, the disease is myelodysplastic syndrome, non-small cell lung cancer, or sickle-cell anemia.

### BRIEF DESCRIPTION OF THE FIGURES

FIG. 1 shows results of a study on the stability of decitabine formulated with cyclodextrins at 2-8° C.

FIG. 2 shows results of a study on the stability of decitabine formulated with cyclodextrins at 25° C.

FIG. 3 shows results of a study on the stability of decitabine in solutions with various HPBCD concentrations at 25° C.

FIG. 4 shows UV spectra of decitabine in cyclodextrin solutions at 25° C.

FIG. 5 shows UV spectra of decitabine in solutions with various HPBCD concentrations at 25° C.

FIG. 6 is a plot of 1/delta A of 262 nm against 1/[HPBCD].

### DETAILED DESCRIPITION OF THE PRESENT INVENTION

The present invention provides improved pharmaceutical formulations of cytidine analogs, e.g., decitabine and azacitidine, which can be used for the treatment of various diseases and conditions, such as myelodysplastic syndrome (MDS), non-small cell lung (NSCL) cancer, and sickle-cell anemia. This innovative approach is taken to overcome three major hurdles that have adversely impacted the commercial development of this type of drugs: hydrolytic degradation in aqueous environment; low solubility in most pharmaceutically acceptable solvents; and minimal oral bioavailability.

As discussed above, in the current clinical formulation decitabine is only slightly soluble in aqueous solutions such as normal saline, and undergoes rapid degradation once dissolved. The instability and limited solubility of decitabine in aqueous solution creates serious inconvenience in both manufacturing and clinical use of decitabine drug product. The instability of dectabine in aqueous solution requires refrigeration of decitabine solution during manufacturing, cold infusion fluids for drug dilution before intravenous administration, and short storage time of decitabine aqueous solutions. The limited solubility in aqueous solution also requires large volume of the drug to be administered, which makes it difficult to administer the drug subcutaneously.

The present invention provides innovative solutions to the above problems associated with the current clinical formulation of decitabine and azacytidine. The inventors discovered that cyclodextrin compounds can significantly increase the solubility of decitabine (or azacytidine) in aqueous solutions, and to dramatically improve its stability in the aqueous solutions. The inventors believe that this invention would minimize or eliminate much of the serious inconvenience in manufacturing, storage and transportation of the decitabine drug product. More importantly, in clinical applications of a drug product of cytotidine analog or derivative (e.g., decitabine, azacytidine, 5-aza-2′-deoxy-2′,2′-difluorocytidine, 5-aza-2′-deoxy-2′-fluorocytidine, gemcitabine, and ara-C) the present invention can reduce patient discomfort and inconvenience by eliminating the need for cold infusion, and lower the volume of the drug solution to be administered, especially for subcutaneous administration.

1. Cyclodextrin in the Formulations of the Present Invention

Accordingly to the present invention, the cyclodextrin compound may be an α-, β-, or γ-cyclodextrin. Cyclodextrins are cyclic oligomers of glucose; these compounds form inclusion complexes with another compound whose molecule can fit into the lipophile-seeking cavities of the cyclodextrin molecule. Specifically, α-cyclodextrin contains six glucopyranose units; β-cyclodextrin contains seven glucopyranose units; and γ-cyclodextrin contains eight glucopyranose units. Typically, a cyclodextrin molecule is believed to form a truncated cone having a core opening of 4.7-5.3 Å, 6.0-6.5 Å and 7.5-8.3 Å in α-, β-, or γ-cyclodextrin respectively.

The cyclodextrin compound may be crystalline and amorphous. Preferably the cyclodextrin compound is amorphous. In general amorphous cyclodextrin is prepared by non-selective additions, especially alkylation of the desired cyclodextrin species. Reactions are carried out to yield mixtures containing a plurality of components thereby preventing crystallization of the cyclodextrin. Various alkylated and hydroxyalkyl-cyclodextrins can be made and of course will vary, depending upon the starting species of cyclodextrin and the addition agent used. Among the amorphous cyclodextrins suitable for compositions according to the invention are hydroxypropyl, hydroxyethyl, glucosyl, maltosyl and maltotriosyl derivatives of β-cyclodextrin, carboxyamidomethyl-β-cyclodextrin, carboxymethyl-β-cyclodextrin, hydroxypropyl-β-cyclodextrin and diethylamino-β-cyclodextrin.

In one embodiment, the cyclodextrin compound is a sulfoalkylether cyclodextrin derivative, preferably mono-, tetra or hepta-substituted β-cyclodextrin sulfobutyl ether sodium salt, and more preferably β-cyclodextrin sulfobutyl ether, 7 sodium salt (CAPTISOL®, Cydex, Inc., Lenexa, Kans.).

In another embodiment, the cyclodextrin compound is a substituted hydroxy-β-cyclodextrin, preferably hydroxypropyl, hydroxyethyl, glucosyl, maltosyl and maltotriosyl derivatives of β-cyclodextrin.

The cyclodextrin compound may be an unmodified or modified α-, β-, or γ-cyclodextrin. However, unmodified or modified α-, β-, or γ-cyclodextrin is less preferred in the compositions according to the invention because the unmodified forms tend to crystallize and are relatively less soluble in aqueous solutions. More preferred for the compositions according to the invention are the α-, β-, or γ-cyclodextrins that are chemically modified or substituted. Chemical substitution at the 2, 3 and 6 hydroxyl groups of the glucopyranose units of the cyclodextrin rings yields increases in solubility of the cyclodextrin compound.

Most preferred cyclodextrins in the compositions according to the invention are amorphous cyclodextrin compounds. By amorphous cyclodextrin is meant non-crystalline mixtures of cyclodextrins wherein the mixture is prepared from α-, β-, or γ-cyclodextrin. In general, the amorphous cyclodextrin is prepared by non-selective alkylation of the desired cyclodextrin species. Suitable alkylation agents for this purpose include but are not limited to propylene oxide, glycidol, iodoacetamide, chloroacetate, and 2-diethylaminoethlychloride. Reactions are carried out to yield mixtures containing a plurality of components thereby preventing crystallization of the cyclodextrin. Various alkylated cyclodextrins can be made and of course will vary, depending upon the starting species of cyclodextrin and the alkylating agent used. Among the amorphous cyclodextrins suitable for compositions according to the invention are hydroxypropyl, hydroxyethyl, glucosyl, maltosyl and maltotriosyl derivatives of β-cyclodextrin, carboxyamidomethyl-β-cyclodextrin, carboxymethyl-β-cyclodextrin, hydroxypropyl-β-cyclodextrin and diethylamino-β-cyclodextrin.

In the compositions according to the invention hydroxypropyl-β-cyclodextrin is preferred although the α- or γ-analogs may also be suitable. The particular alkylated α-, β-, or γ-cyclodextrin to be used with the particular cytidine analog or derivative to form the compositions according to the invention will be selected based on the size of the molecule of the cytidine analog or derivative and the relative size of the cavity of the cyclodextrin compound. As with the unsubstituted cyclodextrins mentioned above, it may be advantageous to use alkylated cyclodextrin having a larger cavity when the composition according to the invention also includes an excipient. The use of a particular α-, β, or γ-cyclodextrin with a particular cytidine analog/derivative, or cytidine analog/derivative and excipient in the compositions according to the invention may of course be optimized based on the effectiveness in of maintaining the particular cytidine analog/derivative in solution.

The composition according to the invention may comprise a mixture of two or more of α-, β-, or γ-cyclodextrin. Preferably, the composition according to the invention will comprise only one of the α-, β-, or γ-cyclodextrins.

2. Preparation of Pharmaceutical Formulations

The compositions of the present invention can be administered by any route, preferably in the form of a pharmaceutical composition adapted to such a route, as illustrated below and are dependent on the condition being treated. The formulations can be, for example, administered orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by a catheter or stent), subcutaneously, intraadiposally, intraarticularly, or intrathecally.

For oral administration, the compositions can be in the form of, for example, a capsule, suspension or liquid. The pharmaceutical composition is preferably made in the form of a dosage unit containing a therapeutically-effective amount of the active ingredient.

Alternatively, the compositions of the present invention can be in powder form, or preferably lyophilized powder form, for reconstitution in the appropriate pharmaceutically acceptable carrier at the time of delivery.

The pharmaceutical compositions can be administered via injection. Formulations for parenteral administration can be in the form of aqueous isotonic sterile injection solutions or suspensions. As described above, these solutions or suspensions can be prepared from sterile powders or granules having cytidine analog/derivative, and also optionally including one or more of the cyclodextrin compound. The powders or granules can be dissolved in water, aqueous buffer or a solvent comprising at least 60% v/v water.

In a preferred embodiment, the cytidine analog/derivative can be formulated into a pharmaceutically acceptable composition comprising the cytidine analog/derivative solvated in an aqueous solution. It is believed that the solubility and/or stability of the cytidine analog/derivative will be improved in such pharmaceutical formulations so that the pharmaceutical formulations may be stored for a prolonged period of time prior to use or require less volume of liquid for injection.

As discussed above, in current clinical treatment with decitabine, to minimize drug decomposition decitabine is supplied as lyophilized powder (together with KH₂PO₄ and NaOH) which is reconstituted with sterile water for injection, and diluted in cold infusion fluids prior to administration. Such a formulation and treatment regimen suffers from a few drawbacks. First, refrigeration of decitabine in cold solution becomes essential, which is burdensome in handling and economically less desirable than a formulation that can sustain storage at higher temperatures. Second, due to rapid decomposition of decitabine in aqueous solution, the reconstituted and diluted decitabine solution may only be infused to a patient for a maximum of 3 hr if the solution has been stored in the refrigerator for less than 7 hr. In addition, infusion of cold fluid can cause great discomfort and pain to the patient, which induces the patient's resistance to such a regimen.

By forming a complex between the cytidine analog/derivative and the cyclodextrin compound in the solution, the pharmaceutical formulations can circumvent the above-listed problems associated with the current clinical treatment with decitabine and 5-azacytidine. The cytidine analog/derivative can be formulated in aqueous solutions containing water in at least 60% vol. of the solvent, optionally at least 80%, or optionally at least 90% vol. of the solvent. These inventive formulations are believed to be more chemically stable than the current clinical formulation.

The relative amounts of the cytidine analog or derivative and cyclodextrin compound will vary depending upon the relative amount of each of the cytidine analog/derivative and the effect of the cyclodextrin compound on the cytidine analog/derivative. In general, the ratio of the weight of the cytidine analog/derivative to the weight of cyclodextrin compound may be in a range between 1:1 and 1:5000. Within this range, the solubility and/or stability of the cytidine analog/derivative will be significantly increased when the ratio of the weight of cytidine analog/derivative to the weight of cyclodextrin compound is in a range between the concentration at which the cytidine analog/derivative will not go into solution at the particular amorphous cyclodextrin concentration and 1:2000. A weight to weight ratio in a range of 1:1 to 1:200 and more preferably in a range of 1:5 to 1:50 of cytidine analog/derivative to cyclodextrin compound are believed to be the most effective for increased solubility and/or stability of the. cytidine analog/derivative. For example, decitabine dissolved in an aqueous solution of cyclodextrin compound in a ratio of between 1:10 and 1:300 (drug:cyclodextrin, wt:wt), and a final concentration of the injection solution of 40 mg/ml of decitabine is expected to significantly increase stability as compared to decitabine in normal saline.

Preferably, if the aqueous solution comprising the cytidine analog/derivative and the cyclodextrin compound is to be administered parenterally, especially via the i.v. route, the cyclodextrin compound will be substantially free of pyrogenic contaminants. Cyclodextrin compound, preferably amorphous hydroxypropyl-β-cyclodextrin may be purchased from a number of vendors including Janssen Pharmaceuticals under the tradename Encapsin. In addition, other forms of amorphous cyclodextrin having different degrees of substitution or glucose residue number are available commercially. A method for the production of hydroxypropyl-β-cyclodextrin is disclosed in Pitha et al., U.S. Pat. No. 4,727,064 which is incorporated herein by reference.

To produce the formulations according to the invention, a pre-weighed amount of hydroxypropyl-β-cyclodextrin compound, which is substantially pyrogen free is placed in a suitable depyrogenated sterile container. Methods for depyrogenation of containers and closure components are well known to those skilled in the art and are fully described in the United States Pharmacopeia 23 (United States Pharmacopeial Convention, Rockville, Md. USA). Generally, depyrogenation is accomplished by exposing the objects to be depyrogenated to temperatures above 250° C. for a period of time sufficient to fully incinerate any organic matter. As measured in U.S.P. Bacterial Endotoxin Units, the formulation will contain no more than 10 Bacterial Endotoxin Units per gram of amorphous cyclodextrin. By substantially pyrogen free is meant that the hydroxypropyl-β-cyclodextrin contains less than 10 U.S.P. bacterial endotoxin units per gram using the U.S.P. method. Preferably, the hydroxypropyl-β-cyclodextrin will contain between 0.1 and 5 U.S.P. bacterial endotoxin units per mg, under conditions specified in the United States Pharmacopeia 23.

Sufficient sterile water or aqueous buffer is added to the substantially pyrogen free amorphous cyclodextrin until the desired concentration of hydroxypropyl-β-cyclodextrin is in solution. To this solution a pre-weighed amount of the cytidine analog/derivative is added with agitation and with additional standing if necessary until it dissolves.

The solution may then be filtered through a sterile 0.2 micron filter into a sterile holding vessel and is subsequently filled in sterile depyrogenated vials, optionally lyophilized, and capped. For products that can be stored for long periods of time, a pharmaceutically acceptable preservative may be added to the solution of cytidine analog/derivative and hydroxypropyl-β-cyclodextrin prior to filtration, filling, optionally lyophilization, and capping or alternatively, may be added sterilely after filtration.

Owing to the enhanced stability, the inventive formulation may be stored and transported at ambient temperature, thereby significantly reducing the cost of handling the drug. Further, the inventive formulation may be conveniently stored for a long time before being administered to the patient. In addition, the inventive formulation may be diluted with regular infusion fluid (without chilling) and administered to a patient at room temperature, thereby avoiding causing patients' discomfort associated with infusion of cold fluid.

The cytidine analog/derivative may be dissolved in a solution of cyclodextrin compound at different concentrations. For example, the formulation may optionally comprise between 0.1 and 200; between 1 and 100; between 1 and 50; between 2 and 50; between 2 and 100; between 5 and 100; between 10 and 100 or between 20 and 100 mg cytidine analog/derivative per ml of the solution. Specific examples of the cytidine analog/derivative per solution concentrations include but are not limited to 2, 5, 10, 20, 22, 25, 30, 40 and 50 mg/ml.

The pharmaceutical formulation may further comprise an acidifying agent added to the formulation in a proportion such that the formulation has a resulting pH between about 4 and 8. The acidifying agent may be an organic acid. Examples of organic acid include, but are not limited to, ascorbic acid, citric acid, tartaric acid, lactic acid, oxalic acid, formic acid, benzene sulphonic acid, benzoic acid, maleic acid, glutamic acid, succinic acid, aspartic acid, diatrizoic acid, and acetic acid. The acidifying agent may also be an inorganic acid, such as hydrochloric acid, sulphuric acid, phosphoric acid, and nitric acid.

It is believed that adding an acidifying agent to the formulation to maintain a relatively neutral pH (e.g., within pH 4-8) facilitates ready dissolution of the cytidine analog/derivative in the solvent and enhances long-term stability of the formulation. In alkaline solution, there is a rapid reversible decomposition of decitabine to N-(formylamidino)-N′-β-D-2-deoxyribofuranosylurea, which decomposes irreversibly to form 1-β-D-2′-deoxyribofuranosyl-3-guanylurea. The first stage of the hydrolytic degradation involves the formation of N-amidinium-N′-(2-deoxy-β-D-erythropentofuranosyl)urea formate (AUF). The second phase of the degradation at an elevated temperature involves formation of guanidine. In acidic solution, N-(formylamidino)-N′-β-D-2-deoxyribofuranosylurea and some unidentified compounds are formed. In strongly acidic solution (at pH <2.2) 5-azacytosine is produced. Thus, maintaining a relative neutral pH may be advantageous for the formulation comprising the cytidine analog/derivative.

In a variation, the acidifying agent is ascorbic acid at a concentration of 0.01-0.2 mg/ml of the solvent, optionally 0.04-0.1 mg/ml or 0.03-0.07 mg/ml of the solvent.

The pH of the pharmaceutical formulation may be adjusted to be between pH 4 and pH 8, preferably between pH 5 and pH 7, and more preferably between pH 5.5 and pH 6.8.

The pharmaceutical formulation is preferably at least 80%, 90%, 95% or more stable upon storage at 2-8° C. for 5, 10, 15, 24 hours, or 2, 4, 7, 14, 21, 28 or more days. The pharmaceutical formulation is also preferably at least 80%, 90%, 95% or more stable upon storage at 20-25° C. for 5, 10, 15, 24 hours, or 2, 5, 7, 14, 21, 28 or more days.

Optionally, steps may be taken to increase the rate at which the cytidine analog/derivative is solvated by the solution containing a cyclodextrin compound. Examples of additional steps that may be performed include, but are nor limited to, agitation, heating, extension of solvation period, and application of micronized cytidine analog/derivative and the combinations thereof.

In one variation, agitation is applied. Examples of agitation include, but are nor limited to, mechanical agitation, sonication, conventional mixing, conventional stirring and the combinations thereof. For example, mechanical agitation of the formulations may be performed according to manufacturer's protocols by Silverson homogenizer manufactured by Silverson Machines Inc., (East Longmeadow, Mass.).

In another variation, heat may be applied. Optionally, the formulations may be heated in a water bath. Preferably, the temperature of the heated formulations may be less than 70° C., more preferably, between 25° C. and 40° C. As an example, the formulation may be heated to 37° C.

In yet another variation, a micronized form of the cytidine analog/derivative may also be employed to enhance solvation kinetics. Optionally, micronization may be performed by a milling process. As an example, micronization may be performed according to manufacturer's protocols by jet milling process performed by Malvern Mastersizer, Mastersizerusing an Air Jet Mill, manufactured by Micron Technology Inc. (Boise, Id.). IncFluid Energy Aljet Inc. (Boise, Id. Telford, Pa.).

Optionally, the pH of the pharmaceutical formulations may be adjusted by commonly used methods. In one variation, pH is adjusted by addition of acid, such as ascorbic acid, or base, such as sodium hydroxide. In another variation, pH is adjusted and stabilized by addition of buffered solutions, such as solution of (Ethylenedinitrilo) tetraacetic acid disodium salt (EDTA). As decitabine and azacitidine are known to be pH-sensitive, adjusting the pH of the pharmaceutical formulations to approximately pH 7 may increase the stability of therapeutic component.

Optionally, separation of non-dissolved cytidine analog/derivative may be performed for the pharmaceutical formulations. Separation may be performed by any suitable technique. For example, a suitable separation method may include one or more of filtration, sedimentation, and centrifugation of the pharmaceutical formulations. Clogging that may be caused by non-dissolved particles of the cytidine analog/derivative, may become an obstacle for administration of the pharmaceutical formulations and a potential hazard for the patient. The separation of non-dissolved cytidine analog/derivative from the pharmaceutical formulations may facilitate administration and enhance safety of the therapeutic product.

Optionally, sterilization of the pharmaceutical formulations may be performed. Sterilization may be performed by any suitable technique. For example, a suitable sterilization method may include one or more of sterile filtration, chemical, irradiation, heat filtration, and addition of a chemical disinfectant to the pharmaceutical formulation.

The pharmaceutical formulation may optionally further include an excipient added in an amount sufficient to enhance the stability of the composition, maintain the product in solution, or prevent side effects (e.g., potential ulceration, vascular irritation or extravasation) associated with the administration of the inventive formulation. Examples of excipients include, but are not limited to, mannitol, sorbitol, lactose, and dextrose.

Optionally, the cytidine analog/derivative may be formulated in an oral dosage form. The formulation is preferably a pharmaceutical composition adapted for oral administration, comprising: a cytidine analog or derivative and a cyclodextrin compound.

The pharmaceutical compositions can be in the form of, for example, a tablet, capsule, liquid or suspension. The pharmaceutical composition is preferably made in the form of a dosage unit containing a therapeutically-effective amount of the cytidine analog or derivative, such as decitabine and 5-azacytidine.

The composition may further comprise binding agents, for example, acacia gum, gelatin, polyvinylpyrrolidone, sorbitol, or tragacanth; glidants; lubricants; fillers, for example, calcium phosphate, glycine, lactose, maize-starch, sorbitol, or sucrose; lubricants, for example, magnesium stearate, polyethylene glycol, silica, or talc; disintegrants, for example, potato starch, flavoring or coloring agents, or acceptable wetting agents; preservatives; coloring agents; flavoring agents; and additives. Examples of additives for include, but are not limited, acacia, almond oil, ethyl alcohol, fractionated coconut oil, gelatin, glucose syrup, glycerin, hydrogenated edible fats, lecithin, methyl cellulose, methyl or propyl para-hydroxybenzoate, propylene glycol, sorbitol, or sorbic acid.

The may be enteric-coated with a coating material. The coating material for enteric-coating of the drug is pH-sensitive and preferably or selectively dissolves at a threshold pH above about 5.2, optionally at pH above about 5.5, optionally at pH above about 5.8, optionally at pH above about 6.0, optionally at pH above about 6.2, optionally at pH above about 6.5, optionally at pH above about 6.5, and most preferably at pH above about 6.8, optionally at pH above about 7.0, optionally at pH above about 7.2, or optionally at pH above about 7.5. The pharmaceutical composition is preferred to substantially disintegrate in an aqueous medium at a pH equal or above the threshold pH within 3 hours, optionally within 2 hours, optionally within 1 hour, more preferably within 30 min, and most preferably within 15 min. The pharmaceutical composition is considered to be substantially disintegrated if at least 50% of the composition disintegrates, e.g., undergoes rupture.

This formulation is believed to protect the drug from decomposition in the gastric juice in the stomach and selectively release the drug in the upper region of the small intestine, preferably in the jejunum, where the pH is slightly acid and close to neutral, which is beyond the threshold pH of the enteric-coat. The disintegration of the enteric-coat leads to selective release of the drug at the specific site of the GI tract where the drug is preferably absorbed, thereby enhancing the oral bioavailability of the drug. In addition, by bypassing decomposition in the stomach, side effects such as damages to the gastric mucosa by the drug and nausea due to stomach irritation can be avoided.

Examples of such a coating material include, but not limited to, cellulose phthalates (e.g, hydropropylmethylcellulose phthaliates (HPMCPs)) that selectively dissolve at pH above 5.6, the Eudragit® family of polymers which are anionic polymer based on methacrylic acid and methacrylates with carboxyl functional groups (e.g., Eudragit L30D with threshold pH of 5.6, Eudragit L with threshold pH of 6.0, and Eudragit S with threshold pH of 6.8), Aquateric with threshold pH of 5.8, polyvinylacetate phthalate (PVAP) that releases drug at pH values above about 5.0, Shellac® that is obtained from a gummy exudation produced by female insects, Laccifer lacca kerr, and releases drug at about pH7.0, and cellulose acetate phthalate (CAP) with threshold pH of 6.0.

In a preferred embodiment, the is enteric-coated with Eudragit L100 with threshold pH of 6.0 or L-100-55 with a threshold pH of 5.5.

Also according to the invention, the pharmaceutical composition is preferred not to substantially disintegrate in an acidic, aqueous medium at pH 1.0-3.0 for at least 1 hour, more preferred not to substantially disintegrate in an acidic, aqueous medium at pH 1.2-2.0 for at least 1 hour, more preferably for at least 2 hours, and most preferably for at least 3 hours. Optionally, the pharmaceutical formulation does not substantially disintegrate in an acidic, aqueous medium at pH 1.2-1.5 for at least 1 hour, more preferably for at least 2 hours, and most preferably for at least 3 hours. The composition is considered to be substantially disintegrated if at least 50% of the composition disintegrates, e.g., undergoes rupture.

In addition, the pharmaceutical composition preferably disintegrates substantially in an aqueous medium at pH 5.2-7.5 within 1 hour, more preferably disintegrates substantially in an aqueous medium at pH 6.0-7.2 within 30 minutes, and most preferably disintegrates substantially in an aqueous medium at pH 6.5-7.0 within 15 minutes. The amount of the enteric-coating material is preferably 1-10% w/w in the composition, more preferably 2-8% w/w in the composition, and most preferably 3-6% w/w in the composition.

The pharmaceutical composition may be in a form of tablet or capsule. In a preferred embodiment, the composition is in a form of tablet. The hardness of the tablet without the enteric-coat is preferably at least 4 kp, more preferably at least 8 kp, and most preferably 10 kp. The size of the tablet is preferably 5-20 mm, more preferably 8-15 mm, and most preferably 10-13 mm.

In any of the above dosage forms, the concentration of the drug is preferably 0.1-20% w/w, more preferably 1-10% w/w, and most preferably 2-5% w/w.

Optionally, the pharmaceutical composition may further comprise a seal-coating material that seals the drug to prevent decomposition due to exposure to moisture, such as hydroxy propylmethylcellulose. Accordingly, the core of the drug is first sealed by the seal-coating material and then coated with the enteric-coating material. This is particularly useful for the formulation of decitabine or 5-azacytidine which is prone to decomposition in exposure to moisture.

Optionally, the pharmaceutical composition may further comprise buffer salt such as potassium or sodium phosphate in an amount sufficient to maintain the pH of the local environment to be 5.2-7.0 when the pharmaceutical composition is dissolved in the GI tract. Examples of such buffer salts include, but are not limited to, KH₂PO₄ and Na₂HPO₄.

Also optionally, the cytidine analog/derivative may be formulated in a topical dosage form. The formulation is preferably a pharmaceutical composition adapted for topical administration, comprising: a cytidine analog or derivative and a cyclodextrin compound.

For topical use the compositions of the present invention can also be prepared in suitable forms to be applied to the skin, or mucus membranes of the nose and throat, and can take the form of creams, ointments, liquid sprays or inhalants, lozenges, or throat paints. Such topical formulations further can include chemical compounds such as dimethylsulfoxide (DMSO) to facilitate surface penetration of the active ingredient.

Also optionally, the cytidine analog/derivative may be formulated in a dosage form suitable for application to the eyes or ears. The cytidine analog/derivative or cyclodextrin compoound can be presented in liquid or semi-liquid form formulated in hydrophobic or hydrophilic bases as ointments, creams, lotions, paints or powders.

Also optionally, the cytidine analog/derivative may be formulated in a dosage form suitable for administration through inhalation. The cytidine analog/derivative or cyclodextrin can be present in liquid sprays or mixed with inhalants, lozenges, or throat paints.

Alternatively, the cytidine analog/derivative or the cyclodextrin compound can be in powder form for reconstitution in the appropriate pharmaceutically acceptable carrier at the time of delivery.

When the cytidine analog/derivative or the cyclodextrin is in solid form. Upon dissolving in liquid, preferably aqueous solution, the cytidine analog/derivative forms complex with the cyclodextrin in the composition. The composition may further comprise buffer salt, which may stabilize the cyclodextrin complex.

3. Vessels or Kits

The cytidine analog/derivative and/or the cyclodextrin compound described in this invention may be contained in a sterilized vessel such as syringe bottles, and glass vials or ampoules of various sizes and capacities. The sterilized vessel may optionally contain the cytidine analog/derivative and/or the cyclodextrin compound in a form of powder or crystalline, or its solution formulation with a volume of e.g., 1-50 ml, 1-25 ml, 1-20 ml or 1-10 ml. Sterilized vessels enable maintain sterility of the pharmaceutical formulations, facilitate transportation and storage, and allow administration of the pharmaceutical formulations without prior sterilization step.

The present invention also provides a kit for administering the cytidine analog/derivative to a host in need thereof. In one embodiment, the kit comprises the cytidine analog/derivative and the cyclodextrin compound in a solid, preferably powder form, and an aqueous diluent that comprises water, buffer, excipient or combinations thereof. Mixing of the solid compounds and the diluent preferably results in the formation of a pharmaceutical formulation according to the present invention. For example, the kit may comprise a first vessel comprising the cytidine analog/derivative, the cyclodextrin compound, and optionally buffer salt (e.g., potassium phosphate), in a solid form; and a vessel container comprising a diluent that comprises water; wherein adding the diluent to the solid compounds results in the formation of a pharmaceutical formulation for administering the cytidine analog/derivative. Mixing the solid compounds and diluent may optionally form a pharmaceutical formulation that comprises between 0.1 and 200 mg of the cytidine analog/derivative per ml of the diluent, optionally between 0.1 and 100, between 2 mg and 50 mg, 5 mg and 30 mg, between 10 mg and 25 mg per ml of the solvent.

In one embodiment, the diluent is injection or infusion fluid. Examples of injection or infusion fluid include, but are not limited to, BWFI (Bacteriostatic Water For Injection), SWFI (Sterile Water For Injection), D5W (Dextrose 5% in Water), D10W (Dextrose 10% in Water), D5LR (Dextrose in Lactate Ringer's Solution), D5¼S (Dextrose 5% in ¼ Strength Saline (5% Dextrose and 0.22% Sodium Chloride Injection)), D5½S (Dextrose 5% in ½ Strength Saline (5% Dextrose and 0.45% Sodium Chloride Injection)), D5NS (Dextrose 5% in Normal Saline (5% Dextrose and 0.9% Sodium Chloride Injection)), D5R (Dextrose 5% in Ringer's Injection), D10NS (Dextrose 10% in Normal Saline (10% Dextrose and 0.9% Sodium Chloride Injection)), IS10W (Invert Sugar 10% in Saline (10% Invert Sugar in 0.9% Sodium Chloride Injection)), LR (Lactated Ringer's Injection), Pr (Protein Hydrolysate Injection), R (Ringer's Injection), NS Sodium Chloride 0.9% (Normal Saline), SOD CL 5 (Sodium Chloride 5% (5% Sodium Chloride Injection), and Sod Lac (Sodium Lactate, ⅙ Molar (M6 Sodium Lactate Injection)).

In another embodiment, the kit comprises the cytidine analog/derivative in a solid, preferably powder form, and a liquid diluent that comprises a cyclodextrin compound dissolved in an aqueous solvent which may comprise water, buffer, excipient or combinations thereof. Mixing of the solid compound and the diluent preferably results in the formation of a pharmaceutical formulation according to the present invention. For example, the kit may comprise a first vessel comprising the cytidine analog/derivative in a solid form; and a vessel container comprising the liquid diluent; wherein adding the diluent to the solid compound results in the formation of a pharmaceutical formulation for administering the cytidine analog/derivative. Mixing the solid compounds and diluent may optionally form a pharmaceutical formulation that comprises between 0.1 and 200 mg of the cytidine analog/derivative per ml of the diluent, optionally between 0.1 and 100, between 2 mg and 50 mg, 5 mg and 30 mg, between 10 mg and 25 mg per ml of the solvent.

The kit may further comprise one or more syringes and/or syringe needles for injecting the pharmaceutical formulation to the patient. For the viscous liquid formulations, syringe needles with high fluidic capacity are preferred, such as DEPOTONE syringe needles (Imprint Pharmaceuticals Ltd., UK).

The kit may optionally further include instructions. The instructions may describe how the solid compounds and the diluent should be mixed to form a pharmaceutical formulation. The instructions may also describe how to administer the resulting pharmaceutical formulation to a patient. It is noted that the instructions may optionally describe the administration methods according to the present invention.

The diluent and the cytidine analog/derivative may be contained in separate vessels. The vessels may come in different sizes. For example, the vessel may comprise between 1 and 50, 1 and 25, 1 and 20, or 1 and 10 ml of the diluent.

The pharmaceutical formulations provided in vessels or kits may be in a form that is suitable for direct administration or may be in a concentrated form that requires dilution relative to what is administered to the patient. For example, pharmaceutical formulations, described in this invention, may be in a form that is suitable for direct administration via infusion.

The methods and kits described herein provide flexibility wherein stability and therapeutic effect of the pharmaceutical formulations comprising the inventive compound may be further enhanced or complemented.

4. Methods of Administration

The cytidine analog/derivative formulated with a cyclodextrin compound can be administered by any route, preferably in the form of a pharmaceutical composition adapted to such a route, as illustrated below and are dependent on the condition being treated. The compounds or formulations can be, for example, administered orally, parenterally, topically, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, or intrathecally. The compounds and/or compositions according to the invention may also be administered or co-administered in slow release dosage forms.

The cytidine analog/derivative formulated with a cyclodextrin compound may be administered or co-administered in any conventional dosage form. Co-administration in the context of this invention is defined to mean the administration of more than one therapeutic agent in the course of a coordinated treatment to achieve an improved clinical outcome. Such co-administration may also be coextensive, that is, occurring during overlapping periods of time.

The cytidine analog/derivative may be administered into a host such as a patient at a dose of 0.1-1000 mg/m², optionally 1-200 mg/m², optionally 1-150 mg/m², optionally 1-100 mg/m², optionally 1-75 mg/m², optionally 1-50 mg/m², optionally 1-40 mg/m², optionally 1-30 mg/m², optionally 1-20 mg/m², or optionally 5-30 mg/m².

For example, the cytidine analog/derivative and the cyclodextrin compound may be supplied as sterile powder for injection, optionally together with buffering salt such as potassium dihydrogen phosphate and pH modifier such as sodium hydroxide. This formulation is preferably stored at 2-25° C., which should keep the drug stable for at least 3 years. This powder formulation may be reconstituted with 10 ml of sterile water for injection. This solution may be further diluted with infusion fluid known in the art, such as 0.9% sodium chloride injection, 5% dextrose injection and lactated ringer's injection, preferably by employing a Y-connector. It is preferred that the reconstituted and diluted solutions be used within 10-11 hours for delivery of maximum potency.

In a preferred embodiment, the cytidine analog/derivative formulated with a cyclodextrin compound is administered to a patient by injection, such as subcutaneous injection, bolus i.v. injection, continuous i.v. infusion and i.v. infusion over 1 hour. Optionally the inventive compound/composition is administered to a patient via an 1-24 hour i.v. infusion per day for 3-5 days per treatment cycle at a dose of 0.1-1000 mg/m² per day, optionally at a dose of 1-200 mg/m² per day, optionally at a dose of 1-150 mg/m² per day, optionally at a dose of 1-100 mg/m² per day, optionally at a dose of 2-50 mg/m² per day, optionally at a dose of 10-30 mg/m² per day, or optionally at a dose of 5-20 mg/m² per day,

For decitabine or azacitidine, the dosage below 50 mg/m² is considered to be much lower than that used in conventional chemotherapy for cancer. By using such a low dose of the analog/derivative of decitabine or azacitidine, transcriptional activity of genes silenced in the cancer cells by aberrant methylation can be activated to trigger downstream signal transduction, leading to cell growth arrest, differentiation and apoptosis, which eventually results in death of these cancer cells. This low dosage, however, should have less systemic cytotoxic effect on normal cells, and thus have fewer side effects on the patient being treated.

The cytidine analog/derivative formulated with a cyclodextrin compound may be co-administered in any conventional form with one or more member selected from the group comprising infusion fluids, therapeutic compounds, nutritious fluids, anti-microbial fluids, buffering and stabilizing agents.

As described above, the cytidine analog/derivative can be formulated with a cyclodextrin compound in a liquid form by solvating the drug and cyclodextrin compound in an aqueous solvent. The pharmaceutical liquid formulations provide the further advantage of being directly administrable, i.e., without further dilution, and thus can be stored in a stable form until administration. Further, because the formulation can be readily mixed with water or infusion/injection fluid, the formulations can be easily and readily further diluted just prior to administration. For example, the pharmaceutical formulations can be diluted with water or infusion/injection fluid 180, 60, 40, 30, 20, 10, 5, 2, 1 minute or less before administration to a patient.

Patients may receive the pharmaceutical formulations parenterally. The preferred route of administration is by intravenous infusion or injection, or by subcutaneous injection. Optionally, the pharmaceutical formulations of the current invention may be infused directly, without prior dilution.

In one embodiment, the pharmaceutical formulation comprising the cytidine analog/derivative and cyclodextrin compound is infused through a connector, such as a Y site connector, that has three arms, each connected to a tube. As an example, Baxter® Y-connectors of various sizes can be used. A vessel containing the pharmaceutical formulation is attached to a tube further attached to one arm of the connector. Infusion fluids, such as 0.9% sodium chloride, or 5% dextrose, or 5% glucose, or Lactated Ringer's, are infused through a tube attached to the other arm of the Y-site connector. The infusion fluids and the pharmaceutical formulations are mixed inside the Y site connector. The resulting mixture is infused into the patient through a tube connected to the third arm of the Y site connector. The advantage of this administration approach over the prior art is that the cytidine analog/derivative is mixed with infusion fluids before it enters the patient's body, thus reducing the time when decomposition of the cytidine analog/derivative may occur due to contact with water. For example, the cytidine analog/derivative is mixed less than 10, 5, 2 or 1 minutes before entering the patient's body.

Patients may be infused with the pharmaceutical formulations for 1, 2, 3, 4, 5 or more hours, as a result of the enhanced stability of the formulations. Prolonged periods of infusion enable flexible schedules of administration of therapeutic formulations.

Alternatively or in addition, speed and volume of the infusion can be regulated according to the patient's needs. The regulation of the infusion of the pharmaceutical formulations can be performed according to existing protocols.

The pharmaceutical formulations may be co-infused in any conventional form with one or more member selected from the group comprising infusion fluids, therapeutic compounds, nutritious fluids, anti-microbial fluids, buffering and stabilizing agents. Optionally, therapeutic components including, but are not limited to, anti-neoplastic agents, alkylating agents, agents that are members of the retinoids superfamily, antibiotic agents, hormonal agents, plant-derived agents, biologic agents, interleukins, interferons, cytokines, immuno-modulating agents, and monoclonal antibodies, may be co-infused with the inventive formulations.

Co-infusion in the context of this invention is defined to mean the infusion of more than one therapeutic agents in a course of coordinated treatment to achieve an improved clinical outcome. Such co-infusion may be simultaneous, overlapping, or sequential. In one particular example, co-infusion of the pharmaceutical formulations and infusion fluids may be performed through Y-type connector.

In humans, decitabine displayed a distribution phase with a half-life of 7 minutes and a terminal half-life on the order of 10-35 minutes as measured by bioassay. The volume of distribution is about 4.6 L/kg. The short plasma half-life is due to rapid inactivation of decitabine by deamination by liver cytidine deaminase. Clearance in humans is high, on the order of 126 mL/min/kg. The mean area under the plasma curve in a total of 5 patients was 408 μg/h/L with a peak plasma concentration of 2.01 μM. In patients decitabine concentrations were about 0.4 μg/ml (2 μM) when administered at 100 mg/m² as a 3-hour infusion. During a longer infusion time (up to 40 hours) plasma concentration was about 0.1 to 0.4 μg/mL. With infusion times of 40-60 hours, at an infusion rate of 1 mg/kg/h, plasma concentrations of 0.43-0.76 μg/mL were achieved. The steady-state plasma concentration at an infusion rate of 1 mg/kg/h is estimated to be 0.2-0.5 μg/mL. The half-life after discontinuing the infusion is 12-20 min. The steady-state plasma concentration of decitabine was estimated to be 0.31-0.39 μg/mL during a 6-hour infusion of 100 mg/m². The range of concentrations during a 600-mg/m² infusion was 0.41-16 μg/mL. Penetration of decitabine into the cerebrospinal fluid in man reaches 14-21% of the plasma concentration at the end of a 36-hour intravenous infusion. Urinary excretion of unchanged decitabine is low, ranging from less than 0.01% to 0.9% of the total dose, and there is no relationship between excretion and dose or plasma drug levels. High clearance values and a total urinary excretion of less than 1% of the administered dose suggest that decitabine is eliminated rapidly and largely by metabolic processes.

Owing to their enhanced stability in comparison with current clinical formulations of decitabine or azacitidine, the inventive formulations comprising a cyclodextrin compound can enjoy longer shelf life when stored and circumvent problems associated with clinical use of decitabine or azacitidine. For example, the cytidine analog/derivative may be supplied as lyophilized powder with a cyclodextrin compound, optionally with acid (e.g., ascorbic acid), alkaline (sodium hydroxide), or buffer salt (monobasic potassium dihydrogen phosphate). The lyophilized powder can be reconstituted with sterile water for injection, e.g., i.v., i.p., i.m., or subcutaneously. Optionally, the powder can be reconstituted with aqueous solvent comprising a water miscible solvent such as glycerin, propylene glycol, ethanol and PEG. The resulting solution may be administered directly to the patient, or diluted further with infusion fluid, such as 0.9% Sodium Chloride; 5% Dextrose; 5% Glucose; and Lactated Ringer's infusion fluid.

The inventive formulations may be stored under ambient conditions or in a controlled environment at 2-25° C. (32-77° F.). In addition, due to the complex formation and enhanced chemical stability, the inventive formulations may be more resistant to enzymatic deamination and hydrolytic degradation, and should have a longer plasma half-life compared to the current clinical formulation of decitabine.

In addition, due to their enhanced chemical stability, the inventive formulations should have a longer plasma half-life compared to the current clinical formulation of decitabine. Thus, the cytidine analog/derivative may be administered to the patient at a lower dose and/or less frequently than that for decitabine or azacitidine.

5. Indications for the Pharmaceutical Formulations and Combination Therapy

The inventive formulations described herein have many therapeutic and prophylactic uses. In a preferred embodiment, the cytidine analog/derivative formulated with a cyclodextrin compound are used in the treatment of a wide variety of diseases that are sensitive to the treatment with a cytidine analog/derivative, such as decitabine or azacitidine. Preferable indications that may be treated using the inventive formulations include those involving undesirable or uncontrolled cell proliferation. Such indications include benign tumors, various types of cancers such as primary tumors and tumor metastasis, restenosis (e.g. coronary, carotid, and cerebral lesions), hematological disorders, abnormal stimulation of endothelial cells (atherosclerosis), insults to body tissue due to surgery, abnormal wound healing, abnormal angiogenesis, diseases that produce fibrosis of tissue, repetitive motion disorders, disorders of tissues that are not highly vascularized, and proliferative responses associated with organ transplants.

Generally, cells in a benign tumor retain their differentiated features and do not divide in a completely uncontrolled manner. A benign tumor is usually localized and nonmetastatic. Specific types benign tumors that can be treated using the present invention include hemangiomas, hepatocellular adenoma, cavernous haemangioma, focal nodular hyperplasia, acoustic neuromas, neurofibroma, bile duct adenoma, bile duct cystanoma, fibroma, lipomas, leiomyomas, mesotheliomas, teratomas, myxomas, nodular regenerative hyperplasia, trachomas and pyogenic granulomas.

In a malignant tumor cells become undifferentiated, do not respond to the body's growth control signals, and multiply in an uncontrolled manner. The malignant tumor is invasive and capable of spreading to distant sites (metastasizing). Malignant tumors are generally divided into two categories: primary and secondary. Primary tumors arise directly from the tissue in which they are found. A secondary tumor, or metastasis, is a tumor which is originated elsewhere in the body but has now spread to a distant organ. The common routes for metastasis are direct growth into adjacent structures, spread through the vascular or lymphatic systems, and tracking along tissue planes and body spaces (peritoneal fluid, cerebrospinal fluid, etc.)

Specific types of cancers or malignant tumors, either primary or secondary, that can be treated using this invention include breast cancer, skin cancer, bone cancer, prostate cancer, liver cancer, lung cancer, brain cancer, cancer of the larynx, gall bladder, pancreas, rectum, parathyroid, thyroid, adrenal, neural tissue, head and neck, colon, stomach, bronchi, kidneys, basal cell carcinoma, squamous cell carcinoma of both ulcerating and papillary type, metastatic skin carcinoma, osteo sarcoma, Ewing's sarcoma, veticulum cell sarcoma, myeloma, giant cell tumor, small-cell lung tumor, gallstones, islet cell tumor, primary brain tumor, acute and chronic lymphocytic and granulocytic tumors, hairy-cell tumor, adenoma, hyperplasia, medullary carcinoma, pheochromocytoma, mucosal neuronms, intestinal ganglloneuromas, hyperplastic corneal nerve tumor, marfanoid habitus tumor, Wilm's tumor, seminoma, ovarian tumor, leiomyomater tumor, cervical dysplasia and in situ carcinoma, neuroblastoma, retinoblastoma, soft tissue sarcoma, malignant carcinoid, topical skin lesion, mycosis fingoide, rhabdomyosarcoma, Kaposi's sarcoma, osteogenic and other sarcoma, malignant hypercalcemia, renal cell tumor, polycythermia vera, adenocarcinoma, glioblastoma multiforma, leukemias, lymphomas, malignant melanomas, epidermoid carcinomas, and other carcinomas and sarcomas.

Hematologic disorders include abnormal growth of blood cells which can lead to dysplastic changes in blood cells and hematologic malignancies such as various leukemias. Examples of hematologic disorders include but are not limited to acute myeloid leukemia, acute promyelocytic leukemia, acute lymphoblastic leukemia, chronic myelogenous leukemia, the myelodysplastic syndromes, and sickle cell anemia.

In some embodiments, the inventive formulations are used to treat blood disorders, including inherited blood disorders and/or disorders where hemoglobin is defective, e.g., sickle cell anemia. In some embodiments, the inventive formulations can be used to treat cancer, including leukemia, pre-leukemia, and other bone marrow-related cancers, e.g., myelodysplatic syndrome (MDS), as well as lung cancer, e.g., non-small cell lung cancer (NSCL). NSCL can include epidermoid or squamous carcinnoma, adenocarcinoma, and large cell carcinoma. MDS can include refractory anemia, refractory anemia with ringed sideroblasts, refractory anemia with excess blasts, refractory anemia with excess blasts in transformation, and chronic myelomonocytic leukemia.

The present invention provides methods, pharmaceutical compositions, and kits for the treatment of animal subjects. The term “animal subject” as used herein includes humans as well as other mammals. The term “treating” as used herein includes achieving a therapeutic benefit and/or a prophylactic benefit. By therapeutic benefit is meant eradication or amelioration of the underlying disorder being treated. For example, in patient with sickle cell anemia, therapeutic benefit includes eradication or amelioration of the underlying sickle cell anemia. Also, a therapeutic benefit is achieved with the eradication or amelioration of one or more of the physiological symptoms associated with the underlying disorder such that an improvement is observed in the patient, notwithstanding the fact that the patient may still be afflicted with the underlying disorder. For example, the inventive formulations provide therapeutic benefit not only when sickle cell anemia is eradicated, but also when an improvement is observed in the patient with respect to other disorders or discomforts that accompany sickle cell anemia, like hand-foot syndrome, fatigue, and or the severity or duration of pain experienced during a crisis (painful episode). Similarly, the inventive formulations can provide therapeutic benefit in ameliorating symptoms associated with cancers, e.g., MDS or NSCL, including anemia, bruising, persistent infections, the size of a lung tumor, and the like.

For prophylactic benefit, the inventive formulations may be administered to a patient at risk of developing a cancer or blood disorder, or to a patient reporting one or more of the physiological symptoms of such a condition, even though a diagnosis of the condition may not have been made.

If necessary or desirable, the inventive formulation may be administered in combination with other therapeutic agents. The choice of therapeutic agents that can be co-administered with the compounds and compositions of the invention will depend, in part, on the condition being treated.

Examples of the therapeutic agent other than the cytidine analog/derivative include, but are not limited to, alkylating agents, antibiotic agents, antimetabolic agents, hormonal agents, plant-derived agents, and biologic agents.

Examples of alkylating agents include, but are not limited to, bischloroethylamines (nitrogen mustards, e.g. chlorambucil, cyclophosphamide, ifosfamide, mechlorethamine, melphalan, uracil mustard), aziridines (e.g. thiotepa), alkyl alkone sulfonates (e.g. busulfan), nitrosoureas (e.g. carmustine, lomustine, streptozocin), nonclassic alkylating agents (altretamine, dacarbazine, and procarbazine), platinum compounds (carboplastin and cisplatin).

Examples of antibiotic agents include, but are not limited to, anthracyclines (e.g. doxorubicin, daunorubicin, epirubicin, idarubicin and anthracenedione), mitomycin C, bleomycin, dactinomycin, plicatomycin.

Examples of antimetabolic agents include, but are not limited to, fluorouracil (5-FU), floxuridine (5-FUdR), methotrexate, leucovorin, hydroxyurea, thioguanine (6-TG), mercaptopurine (6-MP), cytarabine, pentostatin, fludarabine phosphate, cladribine (2-CDA), asparaginase, imatinib mesylate (or GLEEVAC®), and gemcitabine.

Examples of such hormonal agents are synthetic estrogens (e.g. diethylstibestrol), antiestrogens (e.g. tamoxifen, toremifene, fluoxymesterol and raloxifene), antiandrogens (bicalutamide, nilutamide, flutamide), aromatase inhibitors (e.g., aminoglutethimide, anastrozole and tetrazole), ketoconazole, goserelin acetate, leuprolide, megestrol acetate and mifepristone.

Examples of plant-derived agents include, but are not limited to, camptothecins (20(S)-camptothecin, 9-nitro-20(S)-camptothecin, 9-amino-20(S)-camptothecin, irotecan, CPT-11), vinca alkaloids (e.g., vincristine, vinblastine, vindesine, vinzolidine and vinorelbine), podophyllotoxins (e.g., etoposide (VP-16) and teniposide (VM-26)), and taxanes (e.g., paclitaxel and docetaxel).

Examples of biologic agents include, but are not limited to, immuno-modulating proteins such as cytokines, monoclonal antibodies against tumor antigens, tumor suppressor genes, and cancer vaccines. Examples of interleukins that may be used in conjunction with CPT include, but are not limited to, interleukin 2 (IL-2), and interleukin 4 (IL-4), interleukin 12 (IL-12). Examples of interferons that may be used in conjunction with CPT include, but are not limited to, interferon a, interferon b (fibroblast interferon) and interferon g (fibroblast interferon). Examples of such cytokines include, but are not limited to erythropoietin (epoietin a), granulocyte-CSF (filgrastin), and granulocyte, macrophage-CSF (sargramostim). Other immuno-modulating agents other than cytokines include, but are not limited to bacillus Calmette-Guerin, levamisole, and octreotide.

Example of monoclonal antibodies against tumor antigens that can be used in conjunction with CPT include, but are not limited to, HERCEPTIN® (Trastuzumab), RITUXAN® (Rituximab), MYLOTARG® (gemtuzumab ozogamicin), CAMPATH® (alemtuzumab), ZEVALIN® (ibritumomab yiuxetan), PANOREX® (edrecolomab), BEXXAR® (tositumomab), ERBITUX® (cetuximab), and AVASTIN® (bevacizumab).

Examples of the tumor suppressor genes include, but are not limited to, DPC-4, NF-1, NF-2, RB, p 53, WT1, BRCA1 and BRCA2.

Example of cancer vaccines include, but are not limited to gangliosides (GM2), prostate specific antigen (PSA), α-fetoprotein (AFP), carcinoembryonic antigen (CEA) (produced by colon cancers and other adenocarcinomas, e.g. breast, lung, gastric, and pancreas cancer s), melanoma associated antigens (MART-1, gp100, MAGE 1,3 tyrosinase), papillomavirus E6 and E7 fragments, whole cells or portions/lysates of antologous tumor cells and allogeneic tumor cells.

An adjuvant may be used to augment the immune response to TAAs. Examples of adjuvants include, but are not limited to, bacillus Calmette-Guerin (BCG), endotoxin lipopolysaccharides, keyhole limpet hemocyanin (GKLH), interleukin-2 (IL-2), granulocyte-macrophage colony-stimulating factor (GM-CSF) and cytoxan, a chemotherapeutic agent which is believe to reduce tumor-induced suppression when given in low doses.

The present invention also provides a method for treating undesired or uncontrolled angiogenesis. In one embodiment, the method comprises administering to a patient suffering from uncontrolled angiogenesis a therapeutically effective amount of a cytidine analog and derivative in a pharmaceutical formulation of the present invention. The method may further comprise administering to the patient one or more anti-angiogenesis agent.

Examples of anti-angiogenesis agents include, but are not limited to, retinoid acid and derivatives thereof, 2-methoxyestradiol, ANGIOSTATIN™ protein, ENDOSTATIN™ protein, suramin, squalamine, tissue inhibitor of metalloproteinase-I, tissue inhibitor of metalloproteinase-2, plasminogen activator inhibitor-1, plasminogen activator inhibitor-2, cartilage-derived inhibitor, paclitaxel, platelet factor 4, protamine sulphate (clupeine), sulphated chitin derivatives (prepared from queen crab shells), sulphated polysaccharide peptidoglycan complex (sp-pg), staurosporine, modulators of matrix metabolism, including for example, proline analogs ((1-azetidine-2-carboxylic acid (LACA), cishydroxyproline, d,1-3,4-dehydroproline, thiaproline], α,α-dipyridyl, .beta.-aminopropionitrile fumarate, 4-propyl-5-(4-pyridinyl)-2(3h)-oxazolone; methotrexate, mitoxantrone, heparin, interferons, 2 macroglobulin-serum, chimp-3, chymostatin, beta.-cyclodextrin tetradecasulfate, eponemycin; fumagillin, gold sodium thiomalate, d-penicillamine (CDPT), beta.-1-anticollagenase-serum, alpha.2-antiplasmin, bisantrene, lobenzarit disodium, n-(2-carboxyphenyl-4-chloroanthronilic acid disodium or “CCA”, thalidomide; angostatic steroid, cargboxynaminolmidazole; metalloproteinase inhibitors such as BB94. Other anti-angiogenesis agents include antibodies, such as monoclonal antibodies against these angiogenic growth factors: bFGF, aFGF, FGF-5, VEGF isoforms, VEGF-C, HGF/SF and Ang-1/Ang-2.

### EXAMPLES

The following examples are intended to illustrate details of the invention, without thereby limiting it in any manner. As described in the examples below, the use of cyclodextrins as excipients in an aqueous solution can significantly increase the solubility and/or stability of a cytidine analog or derivative such as decitabine and 5-azacytidine.

1. Solubility of Decitabine in Cyclodextrin Solutions

Aqueous solutions of cyclodextrin at pH 6.7-7.2 were prepared by dissolving 4 parts of cyclodextrins (hydroxypropyl α-cyclodextrin (HPACD), hydroxypropyl β-cyclodextrin (HPBCD), β-cyclodextrin (BCD), hydroxypropyl γ-cyclodextrin (HPGCD), or CAPTISOL) in 6 parts of potassium phosphate buffer (50 mM KH₂PO₄, pH 7.0), resulting solutions of 40% w/w cyclodextrins. Solid decitabine (SuperGen, Inc., Dublin, Calif.) was added to the aqueous solution of cyclodextrin and mixed at room temperature (20-25° C.). Table 1 lists the solubility of decitabine in each of the cyclodextrin solutions in comparison with that in the potassium phosphate buffer (pH 7.0).

As shown in Table 1β-cyclodextrins such as HPBCD and Captisol are more effective in enhancing decitabine solubility in aqueous solutions than α-, or γ-cyclodextrins (such as HPACD and HPGCD). The solubility increase would translate into less volume required per dosage clinically, and would make subcutaneous administration of decitabine drug more practical.

TABLE 1

| | KPi | 40% HPBCD | 40% Captisol | 40% HPGCD | 40% HPACD |
|---|---|---|---|---|---|
| | (pH 7.0) | (w/w, pH 7.2) | (w/w, pH 6.7) | (w/w, pH 7.0) | (w/w, pH 7.0) |
| Solubility | 18 | 44 | 42 | 22 | 18 |
| (mg/mL) | | | | | |

b 2. Stability of Decitabine in Cyclodextrin Solutions

The chemical stability of decitabine in the cyclodextrin solutions and in the phophate buffer as prepared above was determined. The initial decitabine concentration for all the solutions was 17-18 mg/mL at the beginning of the experiment at 2-8° C.; 9-10 mg/mL at the beginning of the experiment at 25° C. Decitabine potency was assayed by a HPLC method, and expressed as a percentage of total peak areas at 220 nm.

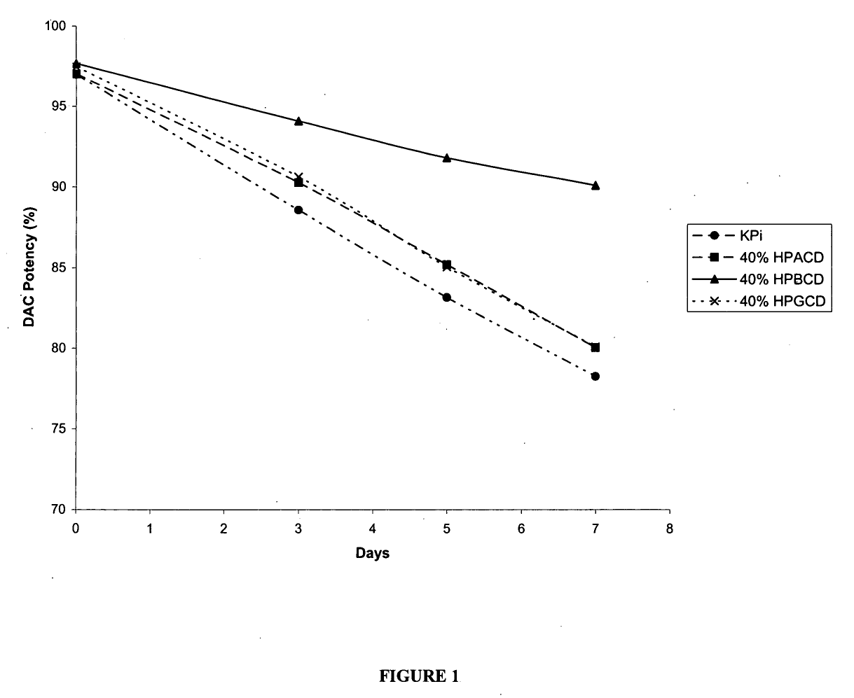

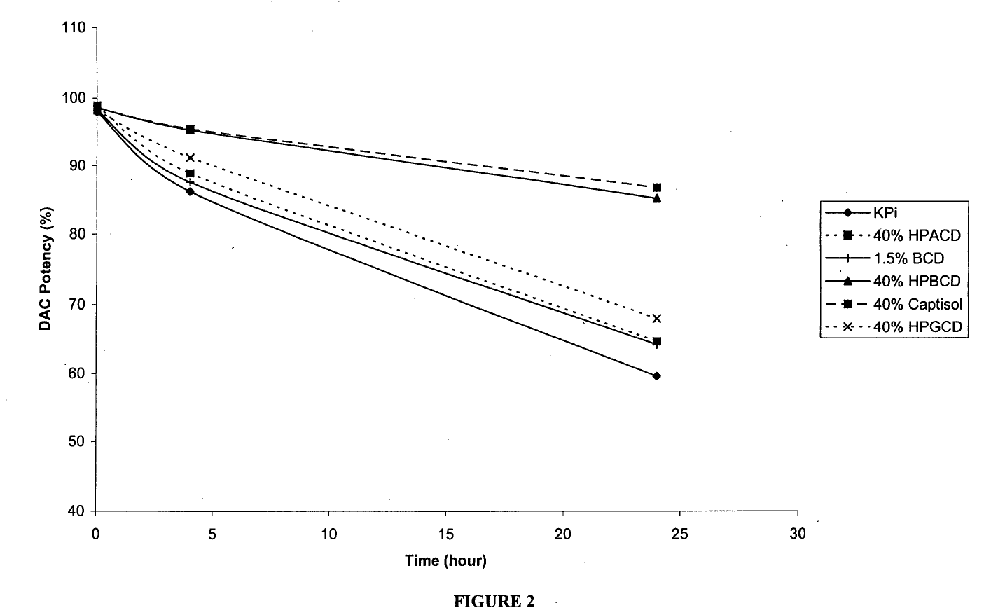

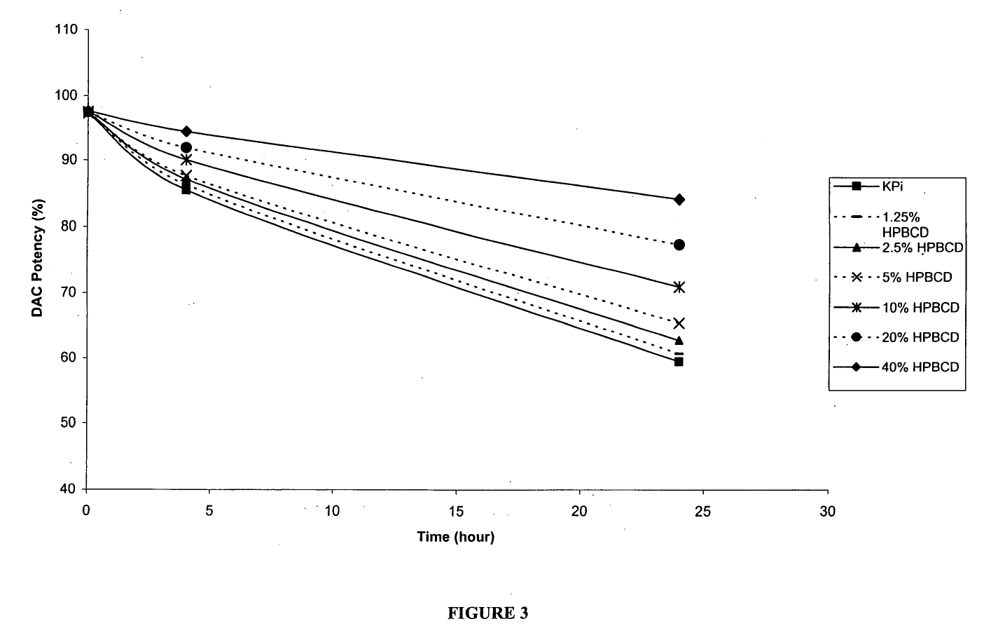

FIGS. 1 and 2 shows that the use of cyclodextrins as excipients in an aqueous solution can dramatically improve the stability of decitabine in the solution both at 2-8° C. and 25° C., respectively. The stabilization effect of cyclodextrins on decitabine in aqueous solutions is more evident when β-cyclodextrins such as HPBCD and Captisol are used, compared to α-, or γ-cyclodextrins (HPACD and HPGCD). This stabilization effect is also concentration dependent, as shown in FIG. 3.

Without being bound to the theory or mechanisms of action, the inventors believe that both the solubility enhancement and stability improvement of decitabine in cyclodextrins—containing aqueous solutions are due to complex formation between decitabine and cyclodextrin molecules, and that β-cyclodextrins form a tighter, reversible complex with decitabine than α-, or γ-cyclodextrins.

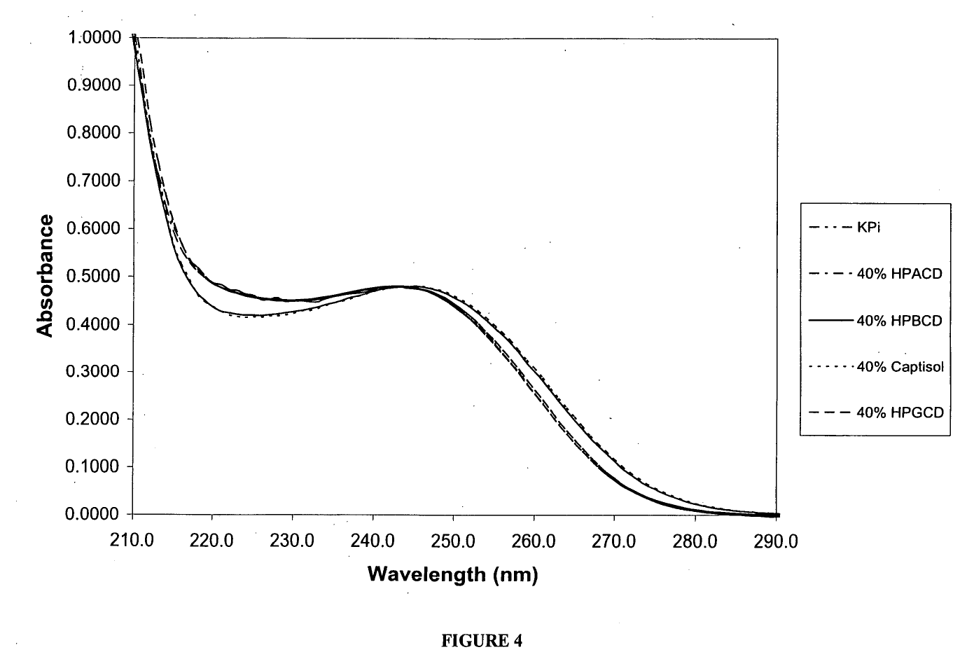

UV spectroscopy study of decitabine in cyclodextrin-containing aqueous solutions was conducted to demonstrate the formation of complex between decitabine and β-cyclodextrins. Each of the spectra was measured at 0.01-0.02 mg/mL of decitabine at room temperature, after blanked with the same solution devoid of decitabine. All of the spectra are normalized to 69 μM of decitabine, assuming the extinction coefficient of decitabine at the apex between 243 nm and 246 nm remains the same (7000 M⁻¹ cm⁻¹). FIG. 4 shows changes of decitabine UV spectrum when β-cyclodextrins are present (FIG. 4), strongly suggesting complex formation between decitabine and β-cyclodextrins.

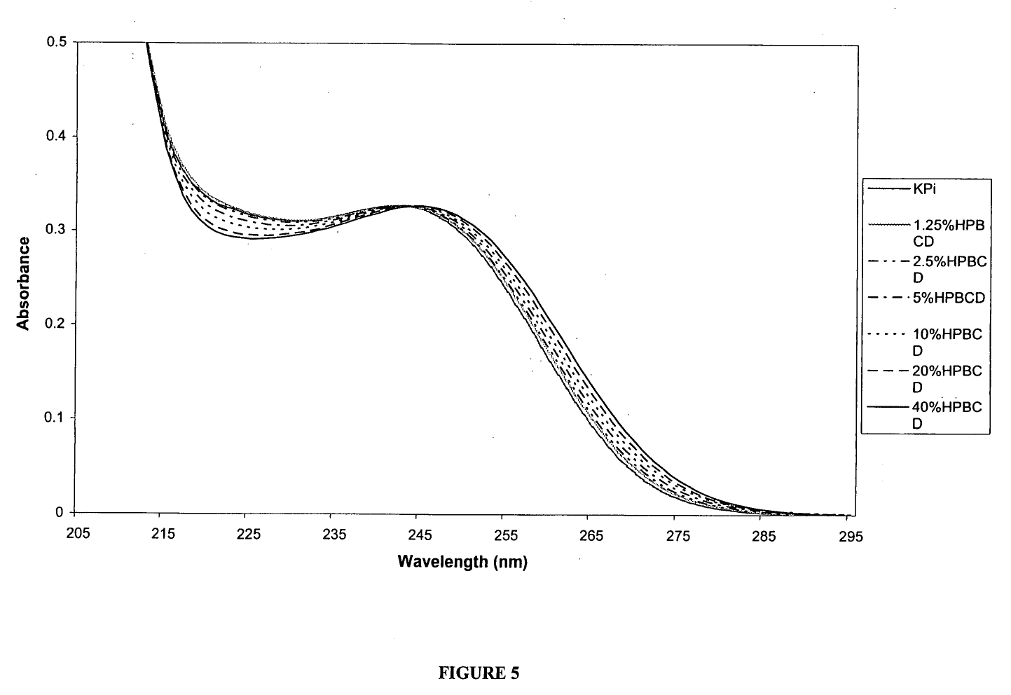

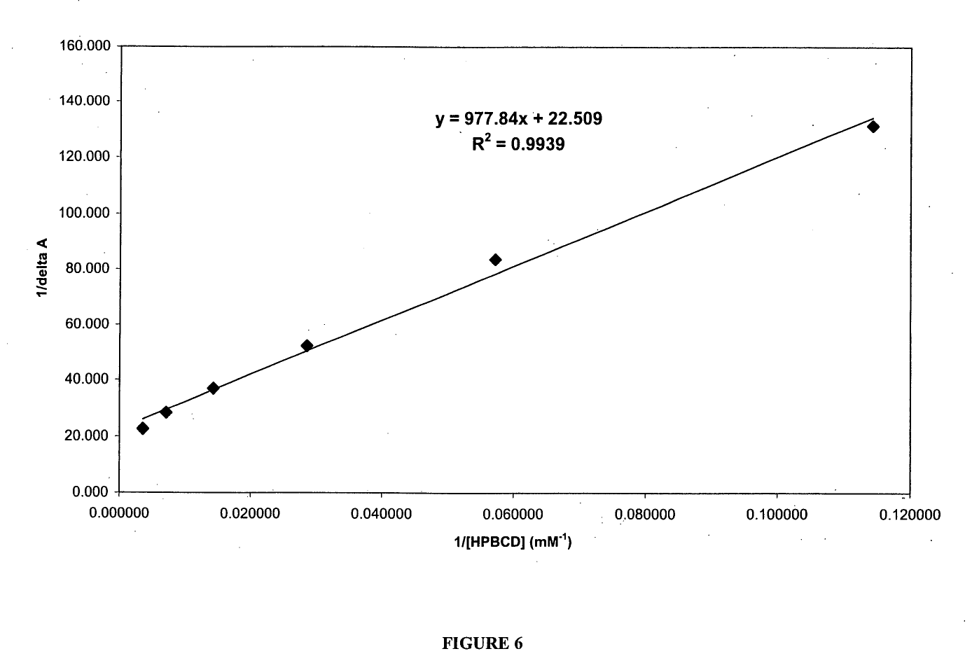

The extent of changes of decitabine UV spectrum of decitabine in cyclodextrin solutions is also shown to be concentration-dependent (FIG. 5). Each of the spectra was measured at 0.01 mg/mL of decitabine at room temperature, after blanked with the same solution devoid of decitabine. All the spectra are normalized to 47 μM of decitabine, assuming the extinction coefficient of decitabine at the apex between 243 nm and 246 mn remains the same (7000 M⁻¹ cm⁻¹).). Based on the adsorbance at 262 nm derived from each of the spectra shown in FIG. 5 and the corresponding HPBCD concentration, the complex formation constant K_{f} for decitabine-HPBCD complex can be calculated based on the following equation (1), assuming the stoichiometry of decitabine/HPBCD complex is 1:1:
1/ΔA=1/(ΔAₒ*K_{f}[HPBCD])+1/ΔAₒ (1)
Here, ΔAₒ is the difference in absorbance at 262 nm between the free and the complexed decitabine, and ΔA is the difference in absorbance at 262 nm between decitabine-HPBCD and decitabine-KPi at the specific [HPBCD] (HPBCD concentration). FIG. 6 shows the plot of 1/ΔA (1/delta A) versus 1/[HPBCD]. In the plot, delta A (ΔA) is the difference in absorbance at 262 nm between decitabine-HPBCD and decitabine-KPi at each specific HPBCD concentration ([HPBCD]). The line is a least-squares fit using equation (1). From the fitting curve, the K_{f} for decitabine-HPBCD complexation is calculated to be 23 M⁻¹.

The effects of α-, or γ-cyclodextrins on both the solubility and stability of decitabine are less dramatic, when compared to β-cyclodextrins such as HPBCD and CAPTISOL, probably due to the formation of less stable complexes between α-, or γ-cyclodextrins and decitabine molecules. The interaction between decitabine and cyclodextrins is believed to be non-covalent and reversible. Under normal clinical drug dosing conditions for decitabine, and based on the calculated K_{f} (23 M⁻¹) for decitabine-HPBCD, it is expected that most of decitabine should dissociate from its complex with HPBCD once infused into patients, and no new drug entity should be formed.

The above results demonstrate that the use of cyclodextrins as excipients in an aqueous solution can increase decitabine solubility in the solution to more than two fold, and also dramatically improve the stability of decitabine in the solution. Cyclodextrins as excipients for decitabine drug can be applied during manufacturing of the drug product, or used in an injectable solution to solubilize the lyophilized decitabine powder just before administration. Delivery of decitabine formulation with cyclodextrins as excipients to a patient may be achieved through all the drug delivery methods, kits, cassettes, and vessels. The great improvement of decitabine solubility and stability in cyclodextrin-containing aqueous solutions would minimize or eliminate a lot of serious inconvenience in both manufacturing and clinical application of current decitabine drug product.

3. Solubility of 5-Azacytidine in Cyclodextrin Solutions

Aqueous solutions of cyclodextrin at pH 6.7-7.2 were prepared by dissolving 4 parts of cyclodextrins (hydroxypropyl α-cyclodextrin (HPACD), hydroxypropyl β-cyclodextrin (HPBCD), hydroxypropyl γ-cyclodextrin (HPGCD), or CAPTISOL) in 6 parts of potassium phosphate buffer (50 mM KH₂PO₄, pH 7.0), resulting solutions of 40% w/w cyclodextrins. Solid 5-azacytidine (Sigma-Aldrich) was added to the aqueous solution of cyclodextrin and mixed at room temperature (20-25° C.). Table 2 lists the solubility of 5-azacytidine in each of the cyclodextrin solutions in comparison with that in the potassium phosphate buffer (pH 7.0).

As shown in Table 2, the use of cyclodextrins as excipients in an aqueous solution can increase 5-azacytidine solubility in the solution. β-cyclodextrins such as HPBCD and Captisol are more effective in enhancing 5-azacytidine solubility in aqueous solutions than α-, or γ-cyclodextrins (such as HPACD and HPGCD). The solubility increase could translate into less volume required per dosage clinically.

TABLE 2

| | KPi | 40% HPBCD | 40% Captisol | 40% HPGCD | 40% HPACD |
|---|---|---|---|---|---|
| | (pH 7.0) | (w/w, pH 7.2) | (w/w, pH 6.7) | (w/w, pH 7.0) | (w/w, pH 7.0) |
| Solubility | 19 | 26 | 26 | 22 | 20 |
| (mg/mL) | | | | | |

It can be appreciated to one of ordinary skill in the art that many changes and modifications can be made to the instant invention without departing from the spirit or scope of the appended claims, and such changes and modifications are contemplated within the scope of the instant invention.

All publications, patents, and patent applications, and web sites are herein incorporated by reference in their entirety to the same extent as if each individual publication, patent, or patent application, was specifically and individually indicated to be incorporated by reference in its entirety.

What is claimed is:

1. A pharmaceutical composition, comprising:
decitabine and a cyclodextrin compound solvated in an aqueous solvent comprising at least 60% v/v water.

2. The pharmaceutical composition of claim 1, wherein decitabine is in the form of pharmaceutically-acceptable salt.

3. The pharmaceutical composition of claim 2, wherein the pharmaceutically-acceptable salt is selected from the group consisting of hydrochloric, hydrobromic, hydroiodic, nitric, carbonic, sulfuric and phosphoric acid.

4. The pharmaceutical composition of claim 2, wherein the pharmaceutically-acceptable salt is selected from the group consisting of aliphatic, cycloaliphatic, aromatic, arylaliphatic, heterocyclic, carboxylic, formic, acetic, propionic, succinic, glycolic, gluconic, maleic, embonic, methanesulfonic, ethanesulfonic, 2-hydroxyethanesulfonic, pantothenic, benzenesulfonic, toluenesulfonic, sulfanilic, mesylic, cyclohexylaminosulfonic, stearic, algenic, β-hydroxybutyric, malonic, galactic, galacturonic acid, L-lactic, acetic, (+)-L-tartaric, citric, butyric, hexanoic, L-aspartic, L-glutamic, succinic, EDTA, maleic, methanesulfonic acid, HBr, HF, HI, nitric, nitrous, sulfuric, sulfurous, phosphorous, perchloric, chloric, chlorous acid, carboxylic acid, sulfonic acid, ascorbic, carbonic, fumaric acid, ethanesulfonic, 2-hydroxyethanesulfonic, and toluenesulfonic acid.

5. The pharmaceutical composition of claim 1, wherein the cyclodextrin compound is amorphous or crystalline.

6. The pharmaceutical composition of claim 1, wherein the cyclodextrin compound is an amorphous α-, β- or γ-cyclodextrin compound.

7. The pharmaceutical composition of claim 1, wherein the cyclodextrin compound is an alkylated or hydroxyalkyl cyclodextrin compound.

8. The pharmaceutical composition of claim 1, wherein the cyclodextrin compound is selected from the group consisting of hydroxypropyl, hydroxyethyl, glucosyl, maltosyl and maltotriosyl derivatives of β-cyclodextrin, carboxyamidomethyl-β-cyclodextrin, carboxymethyl-β-cyclodextrin, hydroxypropyl-β-cyclodextrin and diethylamino-β-cyclodextrin.

9. The pharmaceutical composition of claim 1, wherein the cyclodextrin compound is a sulfoalkylether cyclodextrin derivative.

10. The pharmaceutical composition of claim 1, wherein the cyclodextrin compound is selected from the group consisting of mono-, tetra and hepta-substituted β-cyclodextrin sulfobutyl ether sodium salt.

11. The pharmaceutical composition of claim 1, wherein the cyclodextrin compound is β-cyclodextrin sulfobutyl ether 7 sodium salt or CAPTISOL.

12. The pharmaceutical composition of claim 1, wherein the amount of decitabine in the pharmaceutical composition is between 0.1 and 200 mg per ml of solvent.

13. The pharmaceutical composition of claim 1, wherein the amount of decitabine in the pharmaceutical composition is between 2 and 50 mg per ml of solvent.

14. The pharmaceutical composition of claim 1, wherein the weight ratio of decitabine to the cyclodextrin compound is in a range between 1:1 and 1:5000.

15. The pharmaceutical composition of claim 1, wherein the weight ratio of decitabine to the cyclodextrin compound is in a range between 1:1 and 1:200.

16. The pharmaceutical composition of claim 1, wherein the weight ratio of decitabine to the cyclodextrin compound is in a range between 1:5 and 1:50.

17. The pharmaceutical composition of claim 1, wherein the aqueous solvent comprise at least 90% water.

18. The pharmaceutical composition of claim 1, wherein the aqueous solvent comprise at least 98% water.

19. The pharmaceutical composition of claim 1, wherein the aqueous solvent further comprises propylene glycol, glycerin, polyethylene glycol, or a combination thereof.

20. The pharmaceutical composition of claim 1, further comprising: an acidifying agent added to the composition in a proportion such that the composition has a resulting pH between about 4 and 8.

21. The pharmaceutical composition of claim 20, wherein the acidifying agent is an organic acid.

22. The pharmaceutical composition of claim 21, wherein the organic acid is selected from the group consisting of ascorbic acid, citric acid, tartaric acid, lactic acid, oxalic acid, formic acid, benzene sulphonic acid, benzoic acid, maleic acid, glutamic acid, succinic acid, aspartic acid, diatrizoic acid, and acetic acid.

23. The pharmaceutical composition of claim 20, wherein the acidifying agent is an inorganic acid.

24. The pharmaceutical composition of claim 23, wherein the inorganic acid is selected from the group consisting of hydrochloric acid, sulphuric acid, phosphoric acid, and nitric acid.

25. The pharmaceutical composition of claim 20, wherein the acidifying agent is ascorbic acid at a concentration of 0.01-0.2 mg/ml of the solvent.

26. The pharmaceutical composition of claim 1, further comprising an excipient selected from the group consisting of mannitol, sorbitol, lactose, and dextrose.

27. The pharmaceutical composition of claim 1, wherein the pharmaceutical composition is at least 80% stable upon storage at 2-8° C. for 5, 10, 15, 24 hours, or 2, 4, 7, 14, 21, 28 or more days

28. The pharmaceutical composition of claim 1, wherein the pharmaceutical composition is at least 95% stable upon storage at 2-8° C. for 5, 10, 15, 24 hours, or 2, 4, 7, 14, 21, 28 or more days.

29. The pharmaceutical composition of claim 1, wherein the pharmaceutical composition is at least 80% stable upon storage at 20-25° C. for 5, 10, 15, 24 hours, or 2, 5, 7, 14, 21, 28 or more days.

30. The pharmaceutical composition of claim 1, wherein the pharmaceutical composition is at least 95% stable upon storage at 20-25° C. for 5, 10, 15, 24 hours, or 2, 5, 7, 14, 21, 28 or more days.

31. A pharmaceutical formulation of decitabine prepared by the act comprising:
dissolving decitabine and a cyclodextrin compound in an aqueous solvent that comprises at least 60% v/v water.

32. The pharmaceutical formulation of claim 31, wherein the aqueous solvent further comprises a buffer salt.

33. The pharmaceutical formulation of claim 32, wherein the buffer salt is potassium phosphate.

34. The pharmaceutical formulation of claim 31, the act further comprising: adding an acidifying agent to the solution containing decitabine and the solvent such that pH of the resulting solution is between pH 4 and pH 8.

35. The pharmaceutical formulation of claim 31, the act further comprising: adding an acidifying agent to the solution containing decitabine and the solvent such that pH of the resulting solution is between pH 5.5 and pH 6.8.

36. A sterilized vessel for administering decitabine to a host in need thereof, comprising the pharmaceutical composition of claim 1.

37. The vessel of claim 36, wherein the vessel is a vial, syringe, or ampoule.

38. The vessel of claim 36, wherein the vessel contains between 1 and 50 ml of the pharmaceutical composition.

39. A kit for administering decitabine to a host in need thereof, comprising: decitabine in a solid form, and an aqueous diluent that comprises a cyclodextrin compound and at least 60% v/v water.

40. The kit of claim 39, wherein decitabine is contained in a first container in the kit, and the aqueous diluent is contained in a second container in the kit.

41. The kit of claim 39, further comprising: instruction for using the kit.

42. The kit of claim 41, wherein the instruction contains information of how to administer decitabine to a patient.

43. A kit for administering decitabine to a host in need thereof, comprising:
a first container containing decitabine and a cyclodextrin compound; and a second container containing an aqueous diluent comprising at least 60% water.

44. The kit of claim 43, wherein the aqueous diluent further comprises buffer salt.

45. The kit of claim 43, further comprising: instruction for using the kit.

46. The kit of claim 45, wherein the instruction contains information of how to administer decitabine to a patient.

47. A method for treating a patient having a disease that is sensitive to the treatment with decitabine, comprising:
administering a pharmaceutically effective amount of decitabine in the pharmaceutical composition of claim 1 to the patient.

48. The method of claim 47, wherein the pharmaceutical composition is administered orally, parenterally, topically, intraperitoneally, intravenously, intraarterially, transdermally, sublingually, intramuscularly, rectally, transbuccally, intranasally, liposomally, via inhalation, vaginally, intraoccularly, via local delivery, subcutaneously, intraadiposally, intraarticularly, or intrathecally.

49. The method of claim 47, wherein the pharmaceutical composition is administered intravenously, intramuscularly, or subcutaneously.

50. The method of claim 47, wherein the pharmaceutical composition is further diluted with an aqueous diluent and administered to the patient.

51. The method of claim 50, wherein the aqueous diluent is infusion fluid and the diluted pharmaceutical composition is infused into the patient intravenously.

52. The method of claim 50, wherein the pharmaceutical composition is infused into the patient intravenously via a Y-connector.

53. The method of claim 50, wherein the dilution is performed 10 hr, 2 hr, 1 hr, 30 min, 10 min, 5 min or less before administration to the patient.

54. The method of claim 47, further comprising: administering to the patient a therapeutic agent other than decitabine.

55. The method of claim 54, wherein the therapeutic agent is selected from the group consisting of antimetabolic agents, alkylating agents, retinoid compounds, hormonal agents, plant-derived agents, biologic agents, interleukins, interferons, cytokines, immuno-modulating agents, and monoclonal antibodies.

56. The method of claim 47, wherein the disease is selected from the group consisting of benign tumors, cancer, hematological disorders, atherosclerosis, insults to body tissue due to surgery, abnormal wound healing, abnormal angiogenesis, diseases that produce fibrosis of tissue, repetitive motion disorders, disorders of tissues that are not highly vascularized, and proliferative responses associated with organ transplants.

57. The method of claim 47, wherein the disease is selected from the group consisting of myelodysplastic syndrome, leukemia, malignant tumors, and sickle-cell anemia.

58. A method for treating a patient having a disease that is sensitive to the treatment with decitabine, comprising:
mixing decitabine and a cyclodextrin compound that are in a solid form with an aqueous diluent comprising at least 60% v/v water to form a pharmaceutical formulation; and
administering the pharmaceutical formulation to a patient.

59. The method of claim 58, wherein the pharmaceutical formulation is administered orally, parenterally, topically, intraperitoneally, intravenously, intraarterially, transdermally, sublingually, intramuscularly, rectally, transbuccally, intranasally, liposomally, via inhalation, vaginally, intraoccularly, via local delivery, subcutaneously, intraadiposally, intraarticularly, or intrathecally.

60. A method for treating a patient having a disease that is sensitive to the treatment with decitabine, comprising:
mixing decitabine in a solid form with an aqueous diluent comprising a cyclodextrin compound solvated in at least 60% v/v water to form a pharmaceutical formulation; and
administering the pharmaceutical formulation to a patient.

61. The method of claim 60, wherein the pharmaceutical formulation is administered orally, parenterally, topically, intraperitoneally, intravenously, intraarterially, transdermally, sublingually, intramuscularly, rectally, transbuccally, intranasally, liposomally, via inhalation, vaginally, intraoccularly, via local delivery, subcutaneously, intraadiposally, intraarticularly, or intrathecally.

62. The method of claim 60, wherein the disease is selected from the group consisting of benign tumors, cancer, hematological disorders, atherosclerosis, insults to body tissue due to surgery, abnormal wound healing, abnormal angiogenesis, diseases that produce fibrosis of tissue, repetitive motion disorders, disorders of tissues that are not highly vascularized, and proliferative responses associated with organ transplants.

63. The method of claim 60, wherein the disease is selected from the group consisting of myelodysplastic syndrome, leukemia, malignant tumors, and sickle-cell anemia.

64. The method of claim 60, wherein decitabine is administered into the patient at a dose of 0.1-150 mg/m² per day.

65. The method of claim 60, wherein decitabine is administered into the patient at a dose of 1-50 mg/m² per day.

66. The method of claim 60, wherein decitabine is administered into the patient at a dose of 5-30 mg/m² per day.
